(19) United States
(12) Patent Application Publication
(10) Pub. No.: US 2025/0041332 A1
(43) Pub. Date: Feb. 6, 2025

(54) SYSTEMIC INJECTION OF SUPERHEATED PERFLUOROCARBON EMULSION TO IMPROVE VENTILATION AND REDUCE VENTILATOR-INDUCED LUNG INJURY

(71) Applicant: THE BOARD OF REGENTS OF THE UNIVERSITY OF TEXAS SYSTEM, AUSTIN, TX (US)

(72) Inventors: ROBERT F. MATTREY, DALLAS, TX (US); CAROLINE DE GRACIA LUX, PLANO, TX (US); JACQUES LUX, PLANO, TX (US)

(73) Assignee: THE BOARD OF REGENTS OF THE UNIVERSITY OF TEXAS SYSTEM, AUSTIN, TX (US)

(21) Appl. No.: 18/691,294

(22) PCT Filed: Sep. 13, 2022
(86) PCT No.: PCT/US2022/076368

§ 371 Date: Mar. 12, 2024

Related U.S. Application Data
(60) Provisional application No. 63/293,046, filed on Dec. 22, 2021.
(60) Provisional application No. 63/243,530, filed on Sep. 13, 2021.

(51) Int. Cl. A61K 33/16; A61K 9/00; A61P 11/00

(52) U.S. Cl. CPC A61K 33/16; A61K 9/0019; A61K 9/0073; A61P 11/00

(57) ABSTRACT

Methods of treating or preventing a respiratory disease or condition in a subject in need thereof, inflating the lungs of a subject in need thereof, and reducing the positive-end expiratory pressure (PEEP) needed for mechanical ventilation, the methods comprising administering an effective amount of a composition comprising one or more perfluorocarbons to the subject.

### CROSS-REFERENCE TO RELATED APPLICATIONS

This application claims the benefit of priority to U.S. Provisional Application No. 63/243,530, filed Sep. 13, 2021, and U.S. Provisional Application No. 63/293,046, filed Dec. 22, 2021, each of which is incorporated herein by reference in its entirety.

### BACKGROUND

Human lungs contain over half a billion tiny air sacs called alveoli. The surface area of their lining covers over 1000 sf to adequately oxygenate the blood traveling through their walls. When the chest expands to breathe, a vacuum is created that sucks air in to fill the alveoli, which promptly empty when the chest recoils. In normal lungs, breathing is an effortless task, particularly because the alveoli produce a surfactant that coats the alveolar surface to markedly reduce the pressure needed to fill and empty. When diseased or injured, breathing becomes labored because surfactant function decreases making it difficult to expand the lungs, and hypoxia occurs because protein-rich fluid thickens alveolar walls and fills the air spaces decreasing gas exchange. Acute Lung Injury (ALI), as occurs with smoke and other noxious gas inhalation, and inflammatory injuries, as caused by sepsis, pneumonia, trauma (including, for example, burns, fat embolism, and military combat-related injuries), shock, aspiration, blood transfusion, pancreatitis, and inflammatory storm as occurred, for example, with several viral infections such as coronavirus infection (e.g., a SARS-CoV-2 infection also known as COVID-19), are the most common causes of Acute Respiratory Distress Syndrome (ARDS). When breathing becomes labored and the patient is unable to maintain normal oxygen and carbon dioxide blood levels, assisted ventilation becomes necessary. Mechanical ventilation supports patients with injured lungs by driving gas exchange and positive end-expiratory pressure (PEEP) prevents alveoli from collapsing to ease re-inflation with the next inhalation. Mechanical ventilation may be needed until patients receive and respond to disease-specific therapy. Mechanical ventilation, particularly with PEEP has been clearly linked to adverse effects called Ventilator Induced Lung Injury (VILI). Ventilators push in air to inflate the lungs, and like balloons that are difficult to inflate at first, more pressure is needed to inflate collapsed alveoli. However, because ALI does not affect alveoli equally, positive pressure can over-inflate the less affected alveoli causing them to rupture and create air pockets and pneumothoraxes that cannot empty during expiration, further reducing functional lung volume and worsening gas exchange and requiring the breathing of 100% oxygen, which begins to injure the alveolar lining after a few days. Unfortunately, while mechanical ventilation with PEEP saves lives, it creates a vicious cycle as it increases lung inflammation culminating in cardiovascular issues including reduced venous return and ultimately death. Finally, yet importantly, many ARDS survivors suffer long-term complications as mechanical ventilation often results in long-term cognitive and functional impairment.

Many interventions proposed over the past decades aimed at decreasing the positive pressure needed for mechanical ventilation have been unsuccessful. In the 1990's, it was found that high boiling point perfluorocarbon liquids that can dissolve a large volume of gas could be used to fill the lungs to keep them expanded while maintaining gas exchange. Total or partial filling of the lungs to their functional residual capacity (FRC) improved lung compliance, eased ventilation, and enhanced gas exchange in patients with injured lungs. However, total or partial liquid ventilation (PLV) still required positive pressure mechanical ventilation to ventilate with fluids. Unfortunately, clinical trials with PLV did not demonstrate an improvement when treating ARDS over mechanical ventilation with PEEP. Additionally, the treatment was cumbersome to use, uncomfortable for the patient requiring sedation, and required specialized equipment and was ultimately abandoned. Extra-corporeal membrane oxygenation (ECMO) that provides artificial lung function to allow patients' own lungs to rest and heal, gained acceptance in the treatment of ARDS despite requiring specialized equipment and extensive resources, which became major detractors during the COVID-19 pandemic. Thus, a need exists for improved, preferably non-invasive or minimally-invasive therapeutics to support patients in respiratory distress.

### SUMMARY

Disclosed herein are compositions comprising perfluorocarbons (“perfluorocarbon compositions”) and methods of using the perfluorocarbon compositions to improve lung compliance in order to reduce opening pressures to inflate the lungs, maintain gas exchange and improve respiratory status or function, and/or treat respiratory disorders or dysfunctions in a subject.

In some aspects, provided herein are methods of treating or preventing a respiratory disease or condition in a subject in need thereof. The method involves administering an effective amount of a composition comprising one or more perfluorocarbons to the subject.

In some aspects, provided herein are methods of inflating the lungs of a subject. The method involves administering an effective amount of a composition comprising one or more perfluorocarbons to the subject.

In some aspects, provided herein are methods of reducing the positive-end expiratory pressure (PEEP) for mechanical ventilation of a subject on mechanical ventilation or in need of mechanical ventilation. The method involves administering an effective amount of a composition comprising one or more perfluorocarbons to the subject.

According to further aspects of the aforementioned methods, the composition may be administered via inhalation of perfluorocarbon gas. The perfluorocarbon gas may be delivered via an inhaler. The perfluorocarbon gas may be delivered via a mechanical ventilator. According to alternative aspects of the aforementioned methods, the composition may be administered intravenously. The composition may include nanodroplets each having a perfluorocarbon core comprising the one or more perfluorocarbons.

The nanodroplets may have been formed by high pressure microfluidization or sonication at a temperature at which the perfluorocarbon is in liquid form. The microfluidization may have been performed at a pressure between about 2,000 psi and about 23,000 psi. The composition may include microbubbles each having a perfluorocarbon core comprising the one or more perfluorocarbons.

According to some aspects, the one or more perfluorocarbons may include perfluorobutane (PFB), dodecafluoropentane (DDFP), and/or perfluoropropane (PFP). According to certain aspects, the one or more perfluorocarbons includes a mixture of two or three of perfluorobutane (PFB), perfluoropropane (PFP), and dodecafluoropentane (DDFP).

According to some aspects, the effective amount may be an amount effective to fill the subjects' lungs with perfluorocarbon gas to a volume that is sufficient to improve compliance and/or decrease an opening pressure of the lungs. The effective amount may be an amount effective to fill the subject's lungs to a volume that is at least at residual volume (RV) but not more than a functional residual capacity (FRC) with perfluorocarbon gas. According to certain aspects, the effective amount is an amount effective to fill the subject's lungs to the functional residual capacity (FRC) with the perfluorocarbon gas. According to certain aspects, the effective amount is an amount effective to fill the subject's lungs to the residual volume (RV) with the perfluorocarbon gas. The effective amount may be an amount effective to fill the lungs to a predetermined volume. The composition may include nanodroplets having perfluorobutane (PFB) cores, and the effective amount may be about 1/150 of the predetermined volume. The composition may include nanodroplets having perfluoropropane (PFP) cores, and the effective amount may be about 1/155 of the predetermined volume.

According to some aspects, administering the composition involves administering an initial loading dose of the composition followed by a continuous or intermittent administration of the composition. The continuous or intermittent administration of the composition may be administered at a rate sufficient to substantially replace perfluorocarbon gas losses from the subject's lungs that occur after the initial loading dose of the composition. The continuous and/or intermittent administration of the composition may be administered intravenously. The continuous and/or intermittent administration of the composition may be administered as a gas by inhalation.

According to some aspects, the subject has a respiratory disease or condition. The subject may have acute lung injury (ALI) or acute respiratory distress syndrome (ARDS). The subject may have a coronavirus infection, such as COVID-19. The subject may have or is experiencing one or more of: ALI, ARDS, neonatal respiratory distress syndrome, sudden acute respiratory syndrome (SARS), infectious lung disease, chemical pneumonitis, aspiration pneumonia, traumatic lung injury, pulmonary fibrosis, interstitial pneumonitis, atelectasis, sedation, and long-term bed rest. The subject may have ALI or ARDS caused by one or more of shock, trauma, sepsis, pneumonia, aspiration, burns, major surgery, blood transfusion, pancreatitis, inflammatory storm, and/or severe viral infection, such as coronavirus infection (e.g., COVID-19).

According to some aspects, the method reduces an opening pressure of the lungs of the subject. According to some aspects, the subject has surfactant deficiency and/or dysfunction, and the method improves the surfactant deficiency and/or dysfunction in the subject. According to some aspects, the method increases compliance of alveoli of the subject. According to some aspects, the method causes inflation of the lungs of the subject. According to some aspects, the method reduces or prevents collapse of alveoli at the end of exhalation in the subject. According to some aspects, the method reduces or prevents atelectasis in the subject. According to some aspects, the method reduces or prevents inflammation in the lungs of the subject. According to some aspects, the method reduces or prevents pulmonary edema in the subject. According to some aspects, the method enhances oxygen delivery and/or gas exchange in the alveoli of the subject. According to some aspects, the method decreases ventilation-perfusion mismatch (V-Q mismatch) in the lungs of the subject. According to some aspects, the method improves diffusing capacity (DLCO) in the lungs of the subject. The method may normalize DLCO in the lungs of the subject. According to some aspects, the method improves or normalizes one or more of arterial or venous O₂ concentration (blood O₂ concentration), arterial or venous CO₂ concentration (blood CO₂ concentration), arterial or venous pH (blood pH), and tissue O₂ concentration in the subject. According to some aspects, the method reduces the length of time the subject is on supplemental oxygen or prevents the subject from requiring supplemental oxygen. According to some aspects, the method reduces or prevents one or more symptoms associated with a respiratory disease or condition in the subject. The one or more symptoms may be dyspnea, wheezes, chest tightness, and/or cough. According to some aspects, the method improves exercise tolerance in the subject. According to some aspects, the method reduces the length of time or frequency the subject is hospitalized or admitted to an ICU, or prevents the subject from being hospitalized or admitted to an ICU. According to some aspects, the method reduces FiO₂ needed for adequate oxygenation in the subject.

According to some aspects, the subject is undergoing or in need of mechanical ventilation. According to certain aspects, the subject is on mechanical ventilation at the time of the administration of the composition. According to certain aspects, the subject is not yet on mechanical ventilation at the time of the administration of the composition, but mechanical ventilation is initiated after the administration of the composition. The positive-end expiratory pressure (PEEP) of the initiated mechanical ventilation may be set to zero. According to some aspects, the method may reduce an amount of PEEP needed for adequate oxygenation and ventilation in the subject. According to certain aspects, the method reduces PEEP to zero. The method may involve weaning the subject from mechanical ventilation. The method may mitigate the extent of ventilator-induced lung injury (VILI) or ventilator-associated lung injury (VALI) in a subject. The method may prevent the subject from developing VILI or VALI. The method may reduce the length of time the subject is placed on mechanical ventilation. According to certain aspects, the subject is not on mechanical ventilation at the time of administering the composition, and the method prevents the subject from requiring mechanical ventilation.

### BRIEF DESCRIPTION OF THE DRAWINGS

FIG. 1 schematically depicts a volume spirogram showing different lung volumes and capacities during respiration, and lists exemplary values for the lung volumes and capacities in a typical human male subject.

FIG. 2 depicts the measured lung volume filled by PFB gas in normal rat lungs after infusion of 0 (control), 110 μL, 230 μL or 450 μL of a 7% (v/v) PFB nanodroplet emulsion plotted against the liquid PFB dose per 200 g.

FIG. 3 depicts the measured vital capacities (mL) and weights (g) of six normal rats and a linear regression function relating measured vital capacity to rat weight.

FIG. 4 depicts the measured opening pressure (cm H₂O) required to move air into the lungs of normal rats (200-280 g) that have been lavaged three times with saline after being filled by 0 (control) or ˜1 to 4 mL PFB gas.

FIG. 5 depicts the respiratory frequency (s⁻¹) observed via ultrasound in a rat gradually infused with 1.1 mL of a 7% (v/v) PFB/DDFP (1:1) nanodroplet emulsion.

FIGS. 6A-6B depict photographic images of postmortem lungs in rats that had been infused with 1.1 mL of a 7% (v/v) PFB/DDFP (1:1) nanodroplet emulsion (FIG. 6A) or with 1.1 mL of a 7% (v/v) DDFP nanodroplet emulsion (FIG. 6B).

FIGS. 7A-7B depict changes in lung parameters overtime measured via a FLEXIVENT® ventilator in anesthetized rats after infusion with perfluorocarbon nanodroplet emulsions. FIG. 7A depicts changes in inspiratory capacity (IC) in normal rats infused with PFB nanodroplet emulsions (breathing either 100% air or 60% air with 40% PFB vapor) or a C₃BrF₇ nanodroplet emulsion (breathing 100% air). FIG. 7B depicts changes in inspiratory capacity (IC) and static compliance (Cst) in a normal rat infused with a PFB nanodroplet emulsion breathing gas that is cycled between 100% air and 60% air with 40% PFB vapor.

FIG. 7C depicts changes in inspiratory capacity (IC) and static compliance (Cst) in a bleomycin-injured rat infused with a PFB nanodroplet emulsion and breathing 100% air.

FIG. 8 depicts a representative size distribution measured by nanoparticle tracking analysis of a 7% (v/v) PFB nanodroplet emulsion prepared by sonication according to the methods described herein.

### DETAILED DESCRIPTION

Disclosed herein are compositions comprising perfluorocarbons (“perfluorocarbon compositions”) and methods of using perfluorocarbon compositions to improve respiratory function and/or treat respiratory disorders or dysfunctions. Perfluorocarbons (PFCs) are synthetic fluorinated hydrocarbons comprising only or primarily carbon and fluorine atoms. According to select aspects, the perfluorocarbons used in compositions of the present invention comprise only carbon and fluorine atoms. Perfluorocarbons are both chemically and biologically inert, owing to their very strong intramolecular carbon-fluorine bonds. Liquid perfluorocarbons have low surface tension that mitigates deficient lung surfactant to improve lung compliance when used to fill the alveolar spaces. Gaseous perfluorocarbons also have surface physical activity relevant to lung therapy. Gases, such as oxygen (O₂) and carbon dioxide (CO₂), are highly soluble in both liquid and gaseous perfluorocarbons due to the weak intermolecular interactions between the perfluorocarbon molecules. Accordingly, perfluorocarbons make highly suitable agents for filling alveoli, as they will generally not impede gas exchange

Pulmonary surfactants are mixtures of lipids and proteins that are secreted into the alveolar space by alveolar cells to decrease the surface tension between the air/liquid interfaces in the lung. The presence of surfactants reduces the work of breathing by allowing alveoli to empty with exhalation but easily refill with the next breath with minimal effort. Deficient amounts of pulmonary surfactants in premature infants cause neonatal respiratory distress syndrome, and dysfunctional surfactants, as occurs in injured lungs (e.g., arising from COVID-19), cause alveolar collapse and respiratory distress. Natural surfactant lipids that are made up of predominantly dipalmitoylphosphatidylcholine (DPPC), clump and form solid domains in injured lungs decreasing their surfactant property, and inflammatory proteins that fill injured alveoli adsorb onto and inactivate natural surfactants. Although perfluorocarbon gas itself lacks surfactant quality because it is both extremely hydrophobic and lipophobic, it prevents natural surfactants from forming solid domains and displaces inflammatory proteins to reactivate surfactant function. Therefore, the presence of PFC gas in alveoli at end-expiration not only prevents alveolar collapse easing re-inflation, but also improves surfactant function to decrease the effort of inhalation. Accordingly, perfluorocarbon gases act as a pulmonary surfactant substitute to improve lung compliance and the pressure required to inflate the lungs.

### I. Definitions

It should be noted that whenever a value or range of values of a parameter are recited, it is intended that values and ranges intermediate to the recited values are also intended to be part of this disclosure.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to.” The term “including” does not necessarily imply that additional elements beyond those recited must be present.

The term “about” or “approximately” when referring to a number or a numerical range means that the number or numerical range referred to is an approximation within experimental variability (or within statistical experimental error), and, thus, the number or numerical range may vary from, for example, between 1% and 20% of the stated number or numerical range. In some aspects, “about” indicates a value within 20% of the stated value. In more preferred aspects, “about” indicates a value within 10% of the stated value. In even more preferred aspects, “about” indicates a value within 1% of the stated value.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in aspects of the present invention. Notwithstanding that the numerical ranges and parameters setting forth the broad scope of the invention are approximations, the numerical values set forth in the specific examples are reported as precisely as possible. Any numerical values; however, inherently contain certain errors necessarily resulting from error found in their respective measurements.

The term “at least” prior to a number or series of numbers is understood to include the number adjacent to the term “at least”, and all subsequent numbers or integers that could logically be included, as clear from context. When “at least” is present before a series of numbers or a range, it is understood that “at least” can modify each of the numbers in the series or range.

As used herein, “no more than” or “less than” is understood as the value adjacent to the phrase and logical lower values or integers, as logical from context, to zero (if negative values are not possible). When “no more than” is present before a series of numbers or a range, it is understood that “no more than” can modify each of the numbers in the series or range.

As used herein, “up to” as in “up to 10” is understood as up to and including 10, i.e., 0, 1, 2, 3, 4, 5, 6, 7, 8, 9, or 10, in the context of non-negative integers.

Where a range of values is provided, it is understood that each intervening value (e.g., to the tenth of the unit of the lower limit unless the context clearly dictates otherwise) between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

“Atelectasis” as used herein is collapse of an area of the lung. Atelectasis occurs when the alveoli become deflated or filled with fluid. Common causes of atelectasis include surgery, external chest pressure, airway obstruction, decreased surfactant function, and pleural effusions.

“Compliance” in the context of “pulmonary compliance,” “alveolar compliance,” or “compliance of the alveoli” is the expandability of the lungs or alveoli as calculated by (changes in volume)/(changes in pressure) and may be expressed, for example, in a unit of mL/cm H₂O. Decreased compliance refers to less inflatability in response to a given pressure.

“Diffusing capacity” or “DLCO” as used herein refers to the efficiency of transfer of oxygen from the alveoli into the blood. Various conditions and diseases affect diffusing capacity. For instance, ALI, ARDS, pulmonary edema or fibrosis, and atelectasis may decrease diffusing capacity.

“O₂” refers to oxygen (specifically, molecular oxygen as is found in air).

“CO₂” refers to carbon dioxide.

The “fraction of inspired oxygen”, “FiO₂” is the concentration of oxygen in the inspired gas mixture. FiO₂ of room air, or atmospheric air, is 0.21. In some aspects, subjects experiencing hypoxemia or hypoxia require inhalation of gas that has higher FiO₂ than 0.21 to maintain adequate oxygenation. Gas comprising higher FiO₂ than room air may be delivered to a subject in various methods known in the art, including by supplemental oxygen delivery via nasal cannula, face mask, or by mechanical ventilation.

“Tidal volume” or “TV” as used herein refers to a volume of air breathed in and out during normal breathing.

“Functional residual capacity” or “FRC” as used herein refers to a volume of air remaining in the lung at the end of exhalation of a normal breath.

“Vital capacity” or “VC” as used herein refers to a maximum volume of air a person can inhale when breathing in.

“Residual volume” or “RV” as used herein refers to a volume of gas remaining in the lungs after a person has forcibly exhaled all the air in their lungs.

“Inspiratory reserve volume” or “IRV” as used herein refers to a maximal volume that can be inhaled from the end-inspiratory level during normal breathing.

“Inspiratory capacity” or “IC” as used herein refers to the sum of the inspiratory reserve volume and the tidal volume.

“Mechanical ventilation” as used herein is artificial, or machine-assisted ventilation in which mechanical means are used to assist or replace spontaneous breathing in a subject. Mechanical ventilation may be administered to a subject in need thereof by methods well known in the art, for instance by sedating and paralyzing respiratory muscles of the subject, intubating the subject or placing tracheostomy on the subject, and administering oxygenated gas using a mechanical ventilator via an endotracheal tube, a tracheostomy tube, or face mask as in Continuous positive airway pressure (CPAP). A “mechanical ventilator” as used herein is a life support machine, commonly comprising a power source, controls, sensors/monitors, and safety features, and is configured for sending plain or oxygenated gas into the lungs and allowing gas to exit from the lungs. The goals of mechanical ventilation include maintaining adequate oxygenation and ventilation while minimizing complications associated with mechanical ventilation discussed elsewhere in this disclosure. To achieve such goals, modes of mechanical ventilation (e.g., assist-control mode, control mode) and various parameters of mechanical ventilation including continuous positive pressure (e.g., tidal volume, respiration rate, FiO2, Positive end-expiratory pressure (PEEP)) may be set and adjusted according to methods well known in the art and based on the clinical conditions of the subject by, e.g., using dials and buttons in the control features of a mechanical ventilator connected to the subject's airway.

As used herein, “oxygenation” refers to a process of delivering O₂ from the alveoli to the tissues in order to maintain cellular activity. “Ventilation” refers to a process of transport of air in and out of the alveoli. Arterial blood gas (ABG) analysis can provide data regarding the subject's respiratory status. Blood oxygenation can be measured, for example, by pulse oximetry, blood gas analysis, and/or other methods that are well known in the art. Normal values for ABG parameters are well known in the art. In some aspects, normal value ranges of ABG parameters in humans are approximately as follows: pH: 7.35-7.45; PaCO₂ (arterial partial pressure of carbon dioxide): 35-45 mmHg; PaO₂ (arterial partial pressure of oxygen): >80 mmHg; HCO₃: 21-26 nmol/L (with average of about 24 nmol/L). Atalag et al., Pulmonary Function Tests in Clinical Practice, Springer, 2/e, 2019 at p. 170, herein incorporated by reference in its entirety. In some aspects, hypoxemia (a decreased O₂ concentration in the blood; in some specific aspects, PaO₂ below 80 mmHg) may indicate inadequate oxygenation, among other conditions well known in the art. In some aspects, hypercapnia (an elevated CO₂ in the blood; in some specific aspects, PCO₂ above 45 mmHg) may indicate inadequate ventilation, among other conditions well known in the art.

“Opening pressure,” as used herein in the context of airway opening pressure, is the pressure required to move air into the lower airways and the alveoli. Opening pressure of diseased lungs, e.g., lungs of subjects with ALI or ARDS, may be higher than that of non-diseased lungs due to, for instance, surfactant deficiency and/or decreased compliance. By way of example, studies have reported that opening pressure of non-diseased human lungs to be below 40 cm H₂O, and human ARDS lungs to be approximately 55 cmH₂O. See Cressoni M et al., Intensive Care Med, 2017 May; 43(5):603-611 (doi: 10.1007/s00134-017-4754-8); Tusman G et al., Br J Anaesth 1999; 82: 8-13; Lachmann B, Intensive Care Med 1992; 118: 319-21, each of which is herein incorporated by reference in its entirety.

“Positive end-expiratory pressure” or “PEEP” as used herein refers to extrinsic PEEP, and refers to a pressure support applied to the airway of a subject by an external device, e.g., a ventilator at the end of exhalation. In the context of mechanical ventilation, PEEP is applied to the airway of a subject to mitigate end-expiratory alveolar collapse and/or improve oxygenation. Without wishing to be bound by theory, applying PEEP increases alveolar pressure and alveolar volume. PEEP further increases the surface area by reopening and stabilizing collapsed or unstable alveoli, thereby improving the ventilation-perfusion match and compliance, and reducing dead space and intrapulmonary shunt effect. Benefits of the use of PEEP include that it enables the patient to maintain an adequate PaO₂ at a low and safe concentration of oxygen (<60%), reducing the risk of oxygen toxicity by administration of high concentration of oxygen. Adequate levels of PEEP are determined by clinicians based on the patient's clinical scenario and various factors known in the art. Briefly, PEEP of from about 0 to about 25 cm H₂O have been recommended depending on relevant clinical context. In some aspects, subjects with ALI or ARDS may require higher PEEP than subjects without ALI or ARDS for adequate oxygenation and/or ventilation due to surfactant deficiency and/or decreased compliance of the lungs. Gattinoni L et al., Ann Transl Med. 2017 July; 5(14): 288 (doi: 10.21037/atm.2017.06.64), herein incorporated by reference in its entirety. The amount of PEEP applied to a subject may be set and adjusted in any methods known in the art, for instance by adjusting a dial up or down in said control features of a mechanical ventilator connected to the subject's airway.

A “patient” refers to a subject who shows symptoms and/or complications of a respiratory disease or condition, is under the treatment of a clinician (e.g., a pulmonologist), has been diagnosed as having a respiratory disease or condition, and/or is at a risk of developing a respiratory disease or condition. The term “patient” includes human and veterinary subjects. Any reference to subjects in the present disclosure, should be understood to include the possibility that the subject is a “patient” unless clearly dictated otherwise by context.

A “pharmaceutically acceptable carrier,” as used herein, refers to a carrier or excipient that is suitable for use with the subjects or patients described elsewhere herein (e.g., humans and/or animals) without undue adverse side effects (such as toxicity, irritation, and allergic response) commensurate with a reasonable benefit/risk ratio. It can be a pharmaceutically acceptable solvent, dispersion media, suspending agent or other suitable vehicle, for delivering the perfluorocarbon composition (e.g., a nanodroplet emulsion or microbubble dispersion) to the subject, such as through, for example, intravenous injection. Pharmaceutically acceptable carriers may include any diluents, extenders, preservatives, thickeners, antibacterial agents, antifungal agents, isotonic agents, absorption delaying agents, etc. which are compatible with pharmaceutical administration and are well known in the art.

As used herein, “prevention” or “preventing,” when used in reference to a disease or disorder, refers to a reduction in likelihood of developing a disease or a condition, a reduction in severity of a disease or a condition relative to a population having the same risk factors and not receiving treatment as described herein. The failure to develop a disease or a condition, or a delay in the time to develop signs or symptoms by days, weeks, months, or years is considered effective prevention. Prevention may require administration of more than one dose of the perfluorocarbon composition as described elsewhere herein.

As used herein, a “subject” is an animal, such as a mammal, including a primate (such as a human, a non-human primate, e.g., a monkey, and a chimpanzee), a non-primate (such as a cow, a pig, a horse, a goat, a rabbit, a sheep, a hamster, a guinea pig, a cat, a dog, a rat, or a mouse), or a bird that benefits from the methods according to the present disclosure. In some aspects of the invention, the subject is a human, such as a human in respiratory distress, a human on or in need of mechanical ventilation, or a human having atelectasis. The subject may be a female human. The subject may be a male human. In some aspects, the subject is an adult subject. In other aspects, the subject is a pediatric subject, such as a neonate, an infant, or a child.

As used herein, the terms “treating” or “treatment” refer to a beneficial or desired result, such as reducing at least one sign or symptom, e.g., respiratory distress, in a subject. “Treatment” also refers to prevention of a disease or a condition, or prevention of at least one sign or symptoms associated with the disease or the condition (a prophylactic treatment). “Treatment” can also mean prolonging survival as compared to expected survival in the absence of treatment.

“Ventilation-Perfusion mismatch” or “V-Q mismatch”, as used herein, refers to a condition in which one of oxygen and blood supply is disproportionally decreased or increased compared to the other in an area of the lungs. For instance, V-Q mismatch occurs when one or more areas of the lung receive oxygen but no blood flow, or they receive blood flow but no oxygen.

### II. Perfluorocarbon Compositions

Perfluorocarbon compositions may be a dispersion of a non-continuous perfluorocarbon phase in a continuous non-perfluorocarbon phase (e.g., a liquid-in-liquid emulsion or a gas-in-liquid dispersion). In certain aspects, the continuous phase is aqueous (e.g., water or saline or a mixture of PBS, glycerol and propylene glycol). Perfluorocarbon dispersions may comprise particles encapsulating the perfluorocarbon, which may exist within the particle in a liquid and/or gaseous form. According to certain aspects, the particles may comprise nanodroplets and/or microbubbles. In some implementations, nanodroplets and microbubbles may comprise similar structures with the exception that nanodroplets comprise a liquid perfluorocarbon core and microbubbles comprise a gaseous perfluorocarbon core. Based upon the size of nanodroplets, nanobubbles and microbubbles, different concentrations may be appropriate for a therapeutic composition.

As used herein, “nanodroplet” (“ND”) may refer to a particle formed by a surfactant shell encapsulating a liquid core. According to some aspects, the diameter of the nanodroplet or the average diameter of a nanodroplet composition may be no greater than about 50, 75, 100, 125, 150, 175, 200, 225, 250, 275, 300, 350, 400, 500, 600, 700, 800, 900, or 1,000 nm. According to some aspects, the diameter of the nanodroplet or the average diameter of the nanodroplet composition may be at least about 100, 125, 150, 175, 200, 225, 250, 275, or 300 nm in diameter. The nanodroplet (or the average size of a nanodroplet in a composition) may be of a size sufficiently large enough to prevent extravasation of the droplets from blood vessels. According to some aspects, the diameter of the nanodroplet or the average diameter of a nanodroplet composition may be about 50 nm-100 nm, about 50 nm-200 nm, about 50 nm-300 nm, about 100 nm-200 nm, about 100 nm-300 nm, about 100 nm-400 nm, about 100 nm-500 nm, about 100 nm-1 μm, about 200 nm-1 μm, about 300 nm-1 μm, or about 500 nm-1 μm. Nanodroplet compositions may be produced by any method known in the art. Preferably, nanodroplet compositions are produced by methods that result in concentrated and stable nanodroplets at physiological temperatures (e.g., nanodroplets that do not spontaneously evaporate or significantly change size). According to certain aspects, nanodroplets may be formed by any of the methods described in U.S. Pat. App. Pub. No. 2018/0272012 to de Gracia Lux et al., published Sep. 27, 2018; or de Gracia Lux et al., RSC Adv. 2017; 7(77):48561-48568 (doi: 10.1039/C7RA08971F), each of which is herein incorporated by reference in its entirety. According to some aspects, nanodroplets may be formed by a high energy emulsification method, such as high pressure homogenization/microfluidization or sonication, which surprisingly results in more efficient manufacture.

In some embodiments, PFC (e.g., PFB, PFP, C₂BrF₅, C₃BrF₇) nanoemulsions are prepared by sonication. For example, a first dram vial in dry ice containing an 80:15:5 (v:v:v) PBS/propylene glycol/glycerol excipient solution [or 4:6 (v:v) PBS/propylene glycol excipient solution] and a dry phospholipid film (e.g., comprising 1,2-distearoy 1-sn-glycero-3-phosphocholine DSPC and 1,2-distearoyl-sn-glycero-3-phosphoethanolamine-N-methoxy(polyethyleneglycol)-2000 (DSPE-PEG2000) in a 9:1 molar ratio) can be heated on a 70° C. heating block for 15 min, followed by sonication in a bath sonicator until the solution turned clear. The phospholipid solution is then allowed to cool to room temperature and then cooled in cold bath that has temperature lower than the boiling point of the PFC [e.g., for PFB, an ice-salt bath (between −10 and −12° C.); for PFP, an ethanol-dry ice bath (about −80° C.)] for 5 minutes. A second dram vial in cold bath that has temperature lower than the boiling point of the PFC [e.g., for PFB, an ice-salt bath (between −10 and −12° C.); for PFP, an ethanol-dry ice bath (about −80° C.)] containing liquid PFC and cold phospholipid mixture can be sonicated with the probe sonicator. Any volume of PFC can be mixed with phospholipid at any proportions. For example, PFB of about 50 μL, 100 μL, 150 μL, 200 μL, 300 μL, 400 μL, 500 μL, or more; about 50-100 μL, 100-150 μL, 150-200 μL, 200-300 μL, 300-400 μL, 400-500 μL, or more, e.g., 150-200 μL of PFC can be mixed with phospholipid at a concentration of 1-30% v/v (e.g., 1, 2, 3, 4, 5, 6, 7, 8, 9, 10, 15, 20, 25, or 30% v/v) and sonicated. The PFC-phospholipid mixture is sonicated at a temperature that is lower than the boiling point of the PFC. For instance, the PFC-phospholipid mixture is prepared and sonicated at a temperature that is about 1° C. lower, about 5° C. lower, about 10° C. lower, about 1-2° C. lower, about 2-3° C. lower, about 3-4° C. lower, about 4-5° C. lower, about 5-10° C. lower, more than about 10° C. lower, at least about 1° C. lower, at least about 5° C. lower, or at least about 10° C. lower than the boiling point of the PFC. Any sonication condition can be used, including power range, operation frequency, amplitude, duration, and pulse. For instance, the PFC-phospholipid mixture can be sonicated with about 10-200 kHz (e.g., 10, 20, 30, 40, 50, 60, 70, 80, 90, 100, 150, or 200 kHz) at about 1-2000 Watt (e.g., 1, 10, 20, 50, 100, 200, 300, 400, 500, 1000, 1500, or 1500 Watt), at about 20-80% amplitude (e.g., 20%, 30%, 40%, 50%, 60%, 70%, or 80%), for about 1-1000 active seconds (e.g., 1, 2, 3, 4, 5, 6, 7, 8, 9, 10, 15, 20, 30, 40, 50, 60, 70, 80, 90, 100, 200, 300, 400, 500, 600, 700, 800, 900, 1000 active seconds, or more). “Active seconds” as used herein refer to the total duration of actual sonication, excluding any pauses where sonication is applied in pulse mode. For example, sonication can be applied for 1 second on and 1 second off, 5 seconds on and 5 seconds off, or 10 seconds on and 10 seconds off, for about 1-1000 total active seconds. In a specific embodiment, the PFB/phospholipid mixture is sonicated for 10 seconds at 20% power. The probe tip size can be about 1-10 mm in diameter (e.g., 1, 2, 3, 4, 5, 6, 7, 8, 9, 10, or more than 10 mm in diameter) when sonicating about 150-200 μL of PFC with phospholipid. If a drop of non-encapsulated PFC remained at the bottom of the vial, the mixture can be further sonicated. Following sonication, the resulting PFC emulsion can be collected by centrifugation, filtration, or both, and stored. Any suitable methods of centrifugation and/or filtration can be used. For instance, centrifugation can be performed at 200-1500× g (e.g., about 200× g, 300× g, 400× g, 500× g, 600× g, 700× g, 800× g, 900× g, 1000× g, 1100× g, 1200× g, 1300× g, 1400× g, or 1500× g). Filtration can be performed using a filter having a pore size that is larger than the size of the nanoemulsions but small enough to remove any debris, for example of a pore size of about 0.1-5 μm (e.g., 0.1 μm, 0.22 μm, 0.45 μm, 1 μm, 2 μm, 3 μm, 4 μm, or 5 μm). Superheated perfluorobutane and perfluoropropane nanodroplets fabricated according to the foregoing methods have been shown to be stable in circulation against spontaneous vaporization. According to certain aspects, nanodroplets may be formed by any of the methods described in U.S. Pat. App. Pub. No. 2013/0336891 to Dayton et al., published on Dec. 19, 2013; Sheeran et al., Ultrasound Med Biol. 2011 September; 37(9):1518-30 (doi: 10.1016/j.ultrasmedbio.2011.05.021); Sheeran et al., Biomaterials. 2012-4-33(11):3262-9 (doi: 10.1016/j.biomaterials.2012.01.021), or Sheeran et al., IEEE Trans Ultrason Ferroelectr Freq Control. 2017 January; 64(1):252-263 (doi: 10.1109/TUFFC.2016.2619685), each of which is herein incorporated by reference in its entirety. According to certain aspects, nanodroplet emulsifications prepared directly from liquid perfluorocarbons (e.g., via high energy emulsification methods) may be advantageous (e.g., more easily manufactured). Fabrication of nanodroplet emulsifications via bubble condensation will require much larger volumes of a starting microbubble dispersion to achieve the same amount of liquid perfluorocarbon in an emulsification dose. For example, whereas a 7% (v/v) PFB nanodroplet emulsion, as can be prepared according to the emulsification techniques disclosed herein, can deliver 150 μL of liquid PFB in approximately 2.15 mL total volume, it would require condensation of approximately 22.5 mL of PFB gas to achieve the same volume of liquid PFB. As each 1 μm PFB microbubble should comprise approximately 4.18×10⁻¹² mL of PFB gas and microbubble compositions prepared according to standard procedures and formulations (e.g., having the same phospholipid content as described herein) should yield approximately 30-150 μL of gas per batch (having broad distributions of microbubble sizes), significantly larger volumes of microbubble dispersions would be needed for condensation to achieve the same volumes of liquid perfluorocarbon as can be directly produced via emulsification.

As used herein, “microbubble” (“MB”) may refer to a particle formed by a surfactant shell encapsulating a gas core. According to some aspects, the bubble may be no greater than about 10 μm in diameter. Unless otherwise specified, microbubbles, as used herein, may include bubbles less than 1 μm (i.e. nanobubbles), such as bubbles between, for example, about 50 nm-100 nm, about 50 nm-200 nm, about 50 nm-300 nm, about 100 nm-200 nm, about 100 nm-300 nm, about 100 nm-400 nm, about 100 nm-500 nm, about 100 nm-1 μm, about 200 nm-1 μm, about 300 nm-1 μm, or about 500 nm-1 μm. According to some aspects, the average microbubble size within a microbubble composition is at least about 1, 2, 3, 4, or 5 μm. According to some aspects, the average microbubble size is between approximately 1-10, 1-5, 1-4, 1-3, 1-2, 2-5, 2-4, 2-3, 3-5, or 3-4 μm. The microbubble (or the average size of a microbubble in a composition) may be of a size sufficiently large enough to prevent extravasation of the microbubbles from blood vessels. According to certain aspects, microbubbles may be formed by any of the methods described in U.S. Pat. App. Pub. No. 2013/0336891 to Dayton et al., published on Dec. 19, 2013, which is herein incorporated by reference.

In various implementations, nanodroplets may be converted to microbubbles (e.g., via vaporization) and/or microbubbles may be converted to nanodroplets (e.g., via condensation), particularly through applying temperature and/or pressure changes which induce a phase change of the perfluorocarbon core. Unless dictated otherwise by context, any of the nanodroplets compositions disclosed herein may be converted to microbubbles and/or any of the microbubble compositions disclosed herein may be converted to nanodroplets.

Both nanodroplets and microbubbles generally comprise a surfactant shell which encapsulates the perfluorocarbon core. The surfactant shell may comprise one or more types of molecules which lower the interfacial tension between the perfluorocarbon core and the continuous phase, such as a physiological aqueous environment. This exterior shell may comprise, for example, lipids (e.g., phospholipids), proteins (e.g., albumin), sugars, and/or polymers. According to some aspects, the surfactant shell may comprise lipids, such as phospholipids, which self-align under certain conditions to form a hydrophilic external surface and a lipophilic or hydrophobic internal surface. The phospholipids may comprise any standard phospholipid used in the art for forming microbubbles, nanodroplets, micelles, liposomes, etc. According to some aspects, the phospholipids may comprise diacylglyceride structures, such as phosphatidic acid (phosphatidate) (PA), phosphatidylethanolamine (cephalin) (PE), phosphatidylcholine (lecithin) (PC), phosphatidylserine (PS), and phosphoinositides (e.g., posphatidylinositol (PI), phosphatidylinositol phosphate (PIP), phosphatidylinositol bisphosphate (PIP2) and phosphatidylinositol trisphosphate (PIP3). In some aspects, the phospholipids may comprise phosphosphingolipids, such as ceramide phosphorylcholine (Sphingomyelin) (SPH), ceramide phosphorylethanolamine (Sphingomyelin) (Cer-PE), and ceramide phosphoryllipid. According to some aspects, the phospholipid comprises 1,2-Distearoyl-sn-Glycero-3-Phosphocholine (DSPC) or derivatives thereof. According to some aspects, the phospholipid comprises 1,2-Distearoyl-sn-Glycero-3-Phosphoethanolamine (DSPE) or derivatives thereof. According to some aspects, the surfactant shell may comprise one or more co-surfactants, including, for example, fluorinated surfactants such as semifluorinated alkanes (e.g., CₙF₂ₙ₊₁CₘH₂ₘ₊₁ and more complex architectures). Fluorinated co-surfactants may improve stability of the particle in circulation. Increased stability of perfluorocarbon nanodroplets and microbubbles may be beneficial for the applications described herein (e.g., intravenous administration), as more stable particles may provide more sustained perfluorocarbon release into the lungs. Semifluorinated alkanes are described, for example, in U.S. Pat. App. Pub. No. 2018/0272012 to de Gracia Lux et al. and Bertilla et al., “Semifluorinated Alkanes as Stabilizing Agents of Fluorocarbon Emulsions.” In: Kobayashi K., Tsuchida E., Horinouchi H. (eds) Artificial Oxygen Carrier. Keio University International Symposia for Life Sciences and Medicine, (2005) vol 12. Springer, Tokyo, each of which is herein incorporated by reference in its entirety.

According to some aspects, the surfactant molecules may be coupled to polymer chains, such as poly(ethylene glycol) (i.e. the surfactant shell may be PEGylated). For example, the surfactant molecule may comprise 1,2-Distearoyl-sn-Glycero-3-Phosphoethanolamine-N-[Methoxy(Polyethylene glycol)-2000](DSPE-PEG2k). PEGylation of the particle may improve anti-flocculation/colloidal stability, anti-immunogenicity, hydrophilicity, biocompatibility, and/or in vivo circulation time/bioavailability of the microbubbles. For example, PEGylation may inhibit coalescence and/or Ostwald ripening of nanodroplets and/or microbubbles. PEGylation of the external surface of the particle may also provide favorable conditions (e.g., steric) for performing conjugations which functionalize the particle surface. According to some aspects, the surfactant shell may comprise two or more types of surfactant molecules. According to some aspects, the surfactant shell may comprise three or more types of surfactant molecules.

The perfluorocarbon core comprises one or more perfluorocarbons. “Perfluorocarbon” (PFC) as used herein refers to any hydrocarbons of which hydrogen atoms are substituted by fluorine atoms or other halogens. Perfluorocarbon cores help stabilize the perfluorocarbon particles (e.g., nanodroplets) against dissolution, counteracting the effect of outside pressure (blood pressure and Laplace Pressure), since perfluorocarbons are hydrophobic and not prone to escaping the particle. Any perfluorocarbons can be used in the perfluorocarbon core, compositions, and methods of the present disclosure, including but not limited to two-carbon PFCs [e.g., perfluoroethane (C₂F₆), bromopentafluoroethane (C₂BrF₅)], three-carbon PFCs [e.g., perfluoropropane (PFP, C₃F₈), 1-bromoheptafluoropropane (C₃BrF₇)], and four-carbon PFCs [e.g., perfluorobutane (PFB, C₄F₁₀)]. The one or more perfluorocarbons may comprise octafluoropropane (OFP)/perfluoropropane (PFP), decafluorobutane (DFB)/perfluorobutane (PFB), dodecafluoropentane (DDFP)/perfluoropentane/perflenapent, tetradecafluorohexane/perfluorohexane, hexadecafluoroheptane/perfluoroheptane, octadecafluorodecalin/perfluorodecalin, or perfluoro(2-methyl-3-pentanone) (PFMP). The perfluorocarbon core may comprise one or more fluorocarbons selected from the following: 1,2-bis(F-alkyl)ethenes: 1,2-bis(F-buty)ethenes; 1-F-isopropyl, 2-F-hexylethenes; 1,2-bis(F-hexyl)ethenes; perfluoromethyldecalins: perfluorodimethyldecalins; perfluoromethyl- and dimethyl-adamantanes; perfluoromethyl-, dimethyl- and trimethyl-bicyclo (3,3,1) nonanes and their homologs; perfluoroperhydrophenanthrene; ethers of formulae: (CF₃)₂CFO(CF₂ CF₂)₂OCF(CF₃)₂, (CF₃)₂CFO(CF₂ CF₂)₃OCF(CF₃)₂, (CF₃)₂CFO(CF₂ CF₂)₂F, (CF₃)₂CFO(CF₂ CF₂)₃F, F[CF(CF₃)CF₂O]₂CHFCF₃, [CF₃CF₂CF₂(CF₂)u]₂O with u=1, 3 or 5, and amines N(C₃F₇)₃, N(C₄F₉)₃ and N(C₅F₁₁)₃; perfluoro-N-methylperhydroquinolines and perfluoro-N-methylperhydroisoquinolines; and perfluoralkyl hydrides, such as C₆F₁₃H, C₈F₁₇H, C₈F₁₆H₂ and the halogenated derivatives C₈F₁₆Br₂, and (perflubron), C₆F₁₃CBr₂CH₂Br, 1-bromo 4-perfluoroisopropyl cyclohexane, C₈F₁₆Br₂, and CF₃O(CF₂CF₂O)ᵤCF₂CH₂OH with u=2 or 3. In some specific embodiments, perfluorocarbon used in the perfluorocarbon core, compositions, and methods provided herein has low or very low boiling points (BP), for example PFP (e.g., BP −39° C.), C₂BrF₅(BP −21° C.), PFB (e.g., BP −2° C.), and C₃BrF₇ (e.g., PB+12° C.). One PFC, or a combination of two or more PFCs can be mixed with phospholipids to produce PFC emulsions or PFC compositions provided herein. According to some aspects wherein the dispersion comprises microbubbles, the microbubble core may further comprise non-perfluorocarbon gases such as air, sulfur hexafluoride, and/or nitrogen.

The perfluorocarbon composition may comprise a dispersion (e.g., emulsion of nanodroplets) having an surfactant/emulsifier content (e.g., phospholipids and any additional cosurfactants) that results in a stable emulsion. Perfluorocarbon dispersions are well known in the art and the surfactant content may generally be any amount that is known in the art. According to some aspects, the perfluorocarbon composition may comprise a dispersion having a surfactant content of approximately 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 mg/mL. In some aspects, the dispersion is no greater than approximately 3.5 mg/mL surfactant. In some aspects, the dispersion is at least about 1.0 mg/mL surfactant. In some aspects, the dispersion is between about 1.0 and about 3.5 mg/mL surfactant.

The perfluorocarbon composition may comprise a dispersion (e.g., emulsion of nanodroplets) having a perfluorocarbon content that results in a stable emulsion. Perfluorocarbon dispersions are well known in the art and the perfluorocarbon content may generally be any amount that is known in the art. According to some aspects, the perfluorocarbon composition may comprise a dispersion that is approximately 0.1%, 0.2%, 0.3%, 0.4%, 0.5%, 0.6%, 0.7%, 0.8%, 0.9%, 1%, 2%, 3%, 4%, 5%, 6%, 7%, 8%, 99%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50% (v/v) of the perfluorocarbon. In some aspects, the dispersion is no greater than about 30% (v/v). In some aspects, the dispersion is at least about 0.5% (v/v). In some aspects, the dispersion is between about 0.5% and 30% (v/v). In some aspects, the dispersion is between about 1-10% (v/v). In some aspects, the dispersion is about 7% (v/v). Generally, larger perfluorocarbon contents can be achieved by increasing the concentration of surfactant.

The perfluorocarbon composition may be the end product of a dispersion-forming (e.g., emulsification) process. In some aspects, the continuous liquid phase of the dispersion may be exchanged with another liquid (e.g., via dialysis), diluted, or concentrated (e.g., via centrifugation). In some aspects, additional components may be added to the dispersion, e.g., by mixing the dispersion with additional fluids (e.g., saline). According to various aspects, the dispersion may be prepared in an excipient solution. According to certain aspects, the excipient solution may comprise one or more of the following components: water, saline, PBS, glycerol, propylene glycol, Ringer's solution, and dextrose. For example, the excipient solution may comprise a PBS/propylene glycol/glycerol mixture (e.g., 80:15:5 (v:v:v)) or a PBS/propylene glycol mixture (e.g., 4:6 (v:v)). According to some aspects, the excipient solution may be any solution which will not freeze at the working temperatures. The dispersion may be combined with or prepared in any compatible excipient or pharmaceutically acceptable carrier that is suitable for the particular route of administration. Various pharmaceutically acceptable carriers are well known in the pharmaceutical arts.

### III. Delivery of Perfluorocarbon to Alveoli

The perfluorocarbon compositions described herein may be administered through any suitable route including, but not limited to, intravenous (IV), inhalation, intrapulmonary, or a combination thereof. Other routes of administration may include intranasal, insufflation, intra-arterial, mucosal, other suitable routes of administration as are known in the art, or a combination thereof.

### A. Intravenous Delivery

According to some aspects of the invention, perfluorocarbon compositions are administered to a subject intravenously. All intravenously administered low boiling point perfluorocarbons are eventually evaporated into the alveoli and expelled through the lungs (traversing both walls of the capillary endothelial layer and the alveolar cell as well as the interstitial space between them). Perfluorocarbons having lower boiling points (higher volatility) are expelled into the lungs at a higher rate than perfluorocarbons having higher boiling points. For example, half the dose of a perfluoropentane nanodroplet emulsion (BP=26° C.) is recovered from the exhaled gas in less than 3 min, while that of perfluorooctyl bromide (PFOB) (BP=142° C.) is recovered in approximately 3-12 days (e.g., about 3, 4, 5, 6, 7, 8, 9, 10, 11, 12 days). The perfluorocarbon evaporation rate into alveoli is directly related to the perfluorocarbon's vapor pressure and molecular weight. Low boiling point perfluorocarbon encapsulated in nanodroplets can remain liquid until the nanodroplets reach the alveolar capillaries where the perfluorocarbon core evaporates into and expands alveoli, driven by the large partial pressure gradient between the perfluorocarbon vapor pressure and the alveolar pressure that is at 1 ATM (e.g., approximately 330 kPa for PFB vs. 100 kPa for the alveoli). Again, the rate of vaporization is the fastest for the perfluorocarbon with the lowest boiling point. Advantageously, alveoli filled with perfluorocarbon gas are more compliant than alveoli filled with perfluorocarbon liquid, as in, for example, total or partial liquid ventilation. The delivery of perfluorocarbon gas to the alveoli through the blood stream is advantageous since the alveoli which actually receive blood (perfused) will be those that are inflated/treated with the perfluorocarbon gas and will be ventilated, reducing ventilation perfusion mismatch and improving gas exchange with the blood.

Generally, all perfluoropropane nanodroplets (BP=minus 36.7° C.) and likely all perfluorobutane (PFB) nanodroplets (BP=minus 2° C.) injected intravenously will vaporize into the alveoli during their first pass through alveolar capillaries. Because perfluorocarbons easily mix with air, the presence of the perfluorocarbon gas in the alveoli will dilute inspired oxygen but should not hinder gas exchange. The perfluorocarbon gas may trap air in the alveoli (“air trapping”), providing additional inflation of the alveoli. The volume of trapped air may increase over the course of respiration. However, air trapping, to the extent it occurs, may be reversed by controlling the nature of the inhaled gas as described elsewhere herein. In some aspects, air trapping occurs rapidly in lungs treated with non-brominated PFCs. In some aspects, air trapping can be rapidly reversed by the adding PFBs to the inspired gas. The density of liquid perfluorocarbons and gas perfluorocarbons are much higher than that of body fluids or air, respectively, which makes liquid perfluorocarbons, in particular, especially suitable for reaching the parts of the lung where collapse occurs. Since the perfluorocarbon gas is heavier than air, it will not be exhaled rapidly and will continue to partially inflate the alveoli, at the end of expiration. The perfluorocarbon gas will remain in the alveoli at the end of expiration, maintaining higher lung volume at end of expiration and decreasing the opening pressure required to inflate the lungs, as lungs filled with gas are more compliant. Degree of alveolar inflation at end expiration is a balance between the rate of vaporization from blood and washout from alveoli by exhalation. Because alveoli remain partially inflated at end expiration, they will require less pressure to re-inflate, which decreases opening pressure and improves lung compliance. Therefore, the intravenous delivery of perfluorocarbon gas provides a non-invasive means to improve lung compliance and decrease inflation pressure without compromising gas exchange. Many perfluorocarbon gases, such as PFB, are heavier than air and, therefore, should remain in the alveoli at the end of expiration for multiple respiratory cycles. That is, the perfluorocarbon gas will preferentially fill the lungs' residual volume while the air (comprising expired carbon dioxide) is preferentially exhaled. However, the perfluorocarbon gas will gradually mix with the air and be expelled through respiration. Thus, if prolonged inflation of the alveoli is desired, incremental or continuous infusion of perfluorocarbon gas may be needed to replace the perfluorocarbon gas lost and maintain a lung (e.g., injured lung) in an artificially inflated state.

According to various aspects, the perfluorocarbon core comprises at least one low boiling point perfluorocarbon effective to inflate the alveoli, such as perfluoropropane or perfluorobutane. According to select aspects, the perfluorocarbon core comprises perfluorobutane. According to select aspects, the perfluorocarbon core comprises perfluoropropane. According to select aspects, the perfluorocarbon core comprises a mixture of perfluorobutane and perfluoropropane and/or the perfluorocarbon composition comprises a mixture of particles comprising perfluorobutane cores and perfluoropropane cores. The at least one perfluorocarbon must be volatile enough to rapidly evaporate into the alveoli such that the perfluorocarbon accumulates in the alveoli at a rate relative to the rate of loss through exhalation that is sufficient to inflate the alveoli to the desired volume. The perfluorocarbon may have a rate of transfer (e.g., in liquid form) that is greater than the rate of perfluorocarbon gas loss from the lungs. According to certain aspects, the perfluorocarbon may have a rate of transfer that is at least about 1.1, 1.2, 1.3, 1.4, 1.5, 1.6, 1.7, 1.8, 1.9, 2.0, 3.0, 4.0, 5.0, 10.0, 15, 20, 25, 30, 40, 50, 60, 70, 80, 90, or 100 times the rate of perfluorocarbon gas loss from the lungs. According to certain aspects, at least about 50%, 60%, 70%, 75%, 80%, 85%, 90%, 95%, 96%, 97%, 98%, 99%, or 100% of the perfluorocarbon is expelled into the lungs upon first pass through the subject's circulatory system. According to certain aspects, at least about 50%, 60%, 70%, 75%, 80%, 85%, 90%, 95%, 96%, 97%, 98%, 99%, or 100% of the perfluorocarbon is expelled into the lungs by the second pass through the subject's circulatory system. According to certain aspects, at least about 50%, 60%, 70%, 75%, 80%, 85%, 90%, 95%, 96%, 97%, 98%, 99%, or 100% of the perfluorocarbon is expelled into the lungs by the third pass through the subject's circulatory system. According to certain aspects, at least about 50%, 60%, 70%, 75%, 80%, 85%, 90%, 95%, 96%, 97%, 98%, 99%, or 100% of the perfluorocarbon is expelled into the lungs by about 1, 2, 3, 4, 5, 6, 7, 8, 9, 10, 15, 20, 25, or 30 minutes after intravenous injection. According to certain aspects, the rate of perfluorocarbon transfer is at least about 10, 20, 30, 40, 50, 60, 70, 80, 90, 100, 150, 200, 250, 300, 400, 500, 600, 700, 800, 900, or 1,000 mL of gas per minute.

According to some aspects, the perfluorocarbon has a boiling point less than human body temperature (about 37° C.). Nanodroplets comprising perfluorocarbon cores having boiling points below body temperature are considered superheated and are stabilized by their surfactant shell to prevent spontaneous vaporization upon intravenous infusion. According to some aspects, the perfluorocarbon has a boiling point less than the boiling point of perfluoropentane (about 28° C.). According to some aspects, the perfluorocarbon has a boiling point approximately no greater than the boiling point of perfluorobutane (about −2° C.). According to some aspects, the perfluorocarbon has a boiling point no greater than about 30, 25, 20, 15, 10, 5, 4, 3, 2, 1, 0, −1, −2, −3, −4, or −5° C. According to some aspects, the perfluorocarbon has a boiling point approximately no greater than the boiling point of perfluoropropane (about −37° C.). According to some aspects, the perfluorocarbon has a boiling point no greater than about −5, −10, −15, −20, −25, −30, −35, or −40° C. According to some aspects, the perfluorocarbon has a vapor pressure greater than that of perfluoropentane (about 83.99 kPa at 25° C.). According to some aspects, the perfluorocarbon has a vapor pressure approximately no less than that of perfluorobutane (about 330.3 kPa at 25° C.). According to some aspects, the perfluorocarbon has a vapor pressure approximately no less than that of perfluoropropane (about 792 kPa at 21.1° C.). According to some aspects, the perfluorocarbon has a vapor pressure (at 25° C.) that is at least about 50, 100, 150, 200, 250, 300, 350, 400, 450, 500, 550, 600, 650, 700, 750, 800, 850, 900, 850, or 1,000 kPa.

According to some aspects, the one or more low boiling point perfluorocarbons that are effective to inflate the alveoli may be mixed with other fluorocarbons, including any of those described elsewhere herein. Some fluorocarbons which generally transfer across the alveolar boundary too slowly to effectively inflate the alveoli may be mixed with the inflating perfluorocarbons to increase the stability of the infused nanodroplet or even an infused microbubble. For example, nanodroplets comprising mixtures of perfluoropropane or perfluorobutane with higher boiling point perfluorocarbons may increase the stability of the nanodroplets, preventing spontaneous vaporization in circulation, while also substantially retaining the rapid transfer of the low boiling point component across the alveolar boundary.

According to certain aspects, the perfluorocarbon composition may be gradually infused into the subject. For example, the perfluorocarbon composition may be infused at a rate of approximately 25, 50, 75, 100, 125, 150, 175, 200, 225, 250, 275, 300, 325, 350, 375, 400, 425, 450, 475, or 500 μL/min. In some aspects, the perfluorocarbon composition may be infused at a rate between about 100 and 300 μL/min. In some aspects, the perfluorocarbon composition may be infused at rate of approximately 150 μL/min.

### B. Inhalation Delivery

According to some aspects of the invention, perfluorocarbon gas may be administered directly to the lungs via the subject's airway (e.g., via inhalation) into the alveoli. The perfluorocarbon gas may be any of the perfluorocarbon gases administered intravenously, including, for example, perfluoropropane (PFP), perfluorobutane (PFB), or dodecafluoropentane (DDFP). According to some aspects, the perfluorocarbon gas may be a gas that is too heavy and/or not volatile enough for effective intravenous delivery due to a slow transfer rate across the alveolar barrier. The perfluorocarbon gas may generally be heavier than air. According to some aspects, it is advantageous to administer as heavy as a perfluorocarbon gas as possible which remains in gaseous form within the lungs. The perfluorocarbon gas may generally be a compound which remains gaseous at body temperature (37° C.). The perfluorocarbon gas may generally be a compound which remains gaseous at room temperature (e.g., 15-25° C.). For example, the perfluorocarbon gas may have a boiling point that is no less than about 15, 16, 17, 18 19, 20, 21, 22, 23, 24, 25, 26, 27, 28, 29, 30, 31, 32, 33, 34, 35, 36, or 37° C. According to a specific aspect of the invention, the perfluorocarbon gas is perfluoropentane (bp=28° C.). The perfluorocarbon gas may be administered via any orinasal method of administration known in the art which delivers gaseous compounds to the lungs, including those described elsewhere herein. For example, the perfluorocarbon gas may be administered in one or more doses via an inhaler device, as is known for the treatment of asthma. The perfluorocarbon gas may be administered through a face mask/rebreather mask, a partial rebreather mask, or a non-rebreather mask, as is known in the art for delivering oxygen to a subject, or any other device normally used to deliver oxygen. The perfluorocarbon gas may be delivered via a mechanical ventilator providing assisted breathing to a subject. The perfluorocarbon gas may be mixed with one or more other gases, including, for example, air, oxygen, or nitrogen. The perfluorocarbon gas may be heated (e.g., to body temperature) prior to administration to the subject. The source of the perfluorocarbon gas may store the perfluorocarbon gas in a compressed form.

According to some aspects, the administration of perfluorocarbon gas via inhalation may replace or complement (e.g., simultaneously with, preceding, succeeding, or combinations thereof) the administration of perfluorocarbon compositions through other routes of administration (e.g., intravenous administration) described elsewhere herein. Thus, inhalation of perfluorocarbon gas may be the sole or primary means of inflating alveoli. According to other aspects, the inhalation of perfluorocarbon gas may augment the inflation of alveoli effected through other routes of administration (e.g., intravenous administration of perfluorocarbon compositions). The inhalation of perfluorocarbon gas may slow the elimination rate of perfluorocarbon gas from the lungs. Without being bound by theory, it is expected that the partial pressure difference between the perfluorocarbon (e.g., PFB) nanodroplets and alveolar lumen will decrease.

### IV. Dosage

Determination of the effective amount is preferably made by a clinician, e.g., using parameters or factors known or suspected in the art to affect treatment. Generally, the dose may begin with an amount somewhat less than the optimum dose and then be increased by small increments thereafter until the desired or optimum effect is achieved relative to any negative side effects, as is known in the art. Diagnostic measures or parameters used to characterize efficacy may be those that characterize (e.g., quantify or qualify) symptoms of a disease, progression of a disease, or a therapeutic reaction or response to administration of a therapy. For example, the parameters may characterize the variables of mechanical ventilation (e.g., PEEP, FiO₂) required to maintain adequate oxygenation and ventilation in a subject, or the extent of inflammation in the lung.

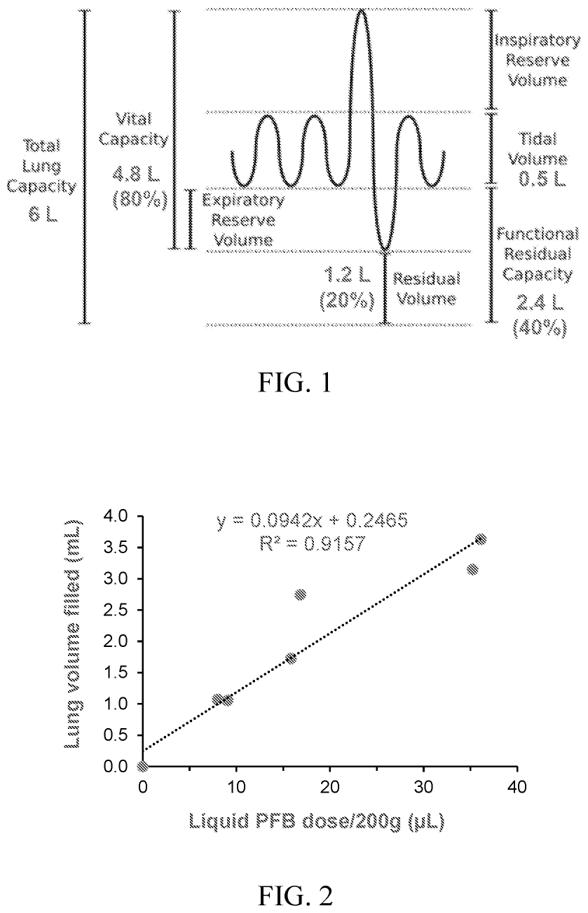

The perfluorocarbon composition may be administered in an amount effective to inflate the subject's lungs by a predetermined volume. In some aspects, the perfluorocarbon composition administered comprises PFP, C₂BrF₅, PFB, C₃BrF₇, or combinations of any thereof. The predetermined volume may be a volume which accounts for any inflation that may be caused by gas osmosis. The volume of trapped air may be correlated to a volume of administered perfluorocarbon (e.g., correlated to a volume of perfluorocarbon gas generated from a known volume of an administered nanodroplet emulsion). FIG. 1 schematically depicts a volume spirogram (available on the world wide web at pathwaymedicine.org/lung-volumes) showing different lung volumes and capacities during respiration, with exemplary values for lung volumes and capacities in a human male. Functional definitions for the different lung volumes are well known in the art. The reference values for lung volume parameters, including total lung capacity (TLC), vital capacity (VC), functional residual capacity (FRC), residual volume (RV), expiratory reserve volume (ERV), tidal volume (TV), and inspiratory reserve volume (IRV) for humans and for reference populations adjusted for one or more of age, sex, race, and height are well known in the art, and can be found for example in Tortora, Gerard J. Principles of anatomy & physiology. Derrickson, Bryan (15th ed.). Hoboken, NJ. p. 874 (ISBN 978-1119447979. OCLC 1020568457), which is herein incorporated by reference in its entirety. The RV, FRC, TLC, or other lung function/volume parameters of the subject may also be determined by performing a pulmonary function test in the subject according to protocols well known in the art, or referring to a pulmonary function test previously performed in the subject. According to some aspects, the predetermined volume is approximately equal to 10%, 20%, 30%, 40%, 50%, 60%, 70%, 75%, 80%, 85%, 90%, 95%, or 100% of the lungs' residual volume (RV). According to some aspects, the predetermined volume is approximately equal to 50%, 60%, 70%, 75%, 80%, 85%, 90%, 95%, or 100% of the lung's functional residual capacity (FRC). According to some aspects, the predetermined volume is approximately no less than the residual volume and approximately no greater than the functional residual capacity. According to some aspects, the predetermined volume is approximately 5%, 10%, 15%, 20%, 25%, 30%, 35%, 40%, 45%, 50%, 55%, or 60% of the subject's total lung capacity. According to some aspects, the predetermined volume is approximately 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9. 2.0, 2.1, 2.2, 2.3, 2.4, 2.5, 2.6, 2.7, 2.8, 2.9, or 3.0 L. According to some aspects, the predetermined volume is between about 0.1-0.5, 0.5-1.0, 1.0-1.5, 1.5-2.0, 2.0-2.5, 0.5-1.5, 0.5-2.0, 0.5-2.5, 1.0-2.0, 1.0-2.5, or 1.5-2.5 L. One or more loading doses may be delivered to a subject which are effective to quickly inflate the lungs by the predetermined volume (e.g., within 1, 2, 3, 4, 5, 6, 7, 8, 9, 10, 15, 20, 25, or 30 min). According to some aspects of the invention, the single PFC loading dose is sufficient to inflate injured lungs to the volume that improves lung compliance and/or decreases work of breathing, and serves as an alternative to application of PEEP.

According to some aspects of the invention, perfluorocarbon may be administered intravenously. The volume of the loading dose of the perfluorocarbon dispersion administered intravenously may be determined based on the predetermined inflation volume. According to some aspects, for relatively fast-transferring compositions (e.g., perfluoropropane or perfluorobutane), it may be assumed that by the time the perfluorocarbon of an intravenously administered dose has been fully expelled into the lungs, only negligible amounts of perfluorocarbon gas have been lost from the lungs through exhalation. Therefore, the predetermined volume may be directly calculated from the volume of the perfluorocarbon in the loading dose of the dispersion. For liquid-in-liquid emulsions, the volume of gas may be calculated from the volume of liquid perfluorocarbon according to the differences in density and the perfluorocarbon concentration in the emulsion. For example, based on the density differences between liquid PFB (1,517 kg/m³) and gaseous PFB (9.9 kg/m³) (i.e., approximately 150-fold difference), intravenous administration of 1 mL liquid PFB given as an emulsion produces approximately 150 mL of expelled PFB gas. Accordingly, for instance, to fill the residual volume in a man of an average height and age, who is according to some aspects expected to have about a 1.2 L residual volume, a nanodroplet emulsion volume containing approximately 8 mL of liquid PFB may be administered intravenously to the subject to inflate the lungs. The dose of liquid PFP would be expected to be slightly lower since PFP volume expands 155 fold when vaporized—density of liquid PFP (1,601 kg/m³) is about 155 fold greater than gaseous PFP (10.3 kg/m³). In some aspects of the invention, nanodroplet compositions may be preferable to microbubble compositions, at least with respect to the loading dose, since microbubbles filled with only PFC gas can only deliver the gas volume carried in their core (e.g., 1-10 μm in size). According to certain aspects of the invention, larger volumes of microbubble compositions may be intravenously administered gradually over longer periods of time relative to nanodroplet compositions to achieve the same inflation volume as the nanodroplet composition. In some instances, a nanodroplet loading dose may be administered to the subject followed by continuous or incremental infusion of a microbubble composition to maintain the inflation volume.

According to some aspects, perfluorocarbon gas may be administered directly to the lungs via the subject's airway (e.g., via inhalation) into the lung. The perfluorocarbon gas may be administered in a volume approximately equal to the predetermined volume described with respect to intravenous administration or may be administered at larger volumes to account for enhanced perfluorocarbon gas loss through exhalation (e.g., loss of the perfluorocarbon gas through exhalation before all of the gas is delivered to alveoli). For example, the volume of perfluorocarbon gas administered into the airway may be approximately 100, 105, 110, 115, 120, 125, 130, 140, 150, 160, 170, 180, 190, 200, 225, 250, 275, 300% or more of the predetermined volume. In some aspects of the invention, nanodroplet compositions may be preferable to airway delivery, at least with respect to the loading dose, because nanodroplets will fill the residual volume more rapidly and will preferentially inflate perfused alveoli. According to certain aspects of the invention, subjects may be given a nanodroplet loading dose to inflate the lungs to a desired volume followed by the inhalation of various mixtures of PFC gas, oxygen and nitrogen via the airways over longer periods of time relative to nanodroplet compositions to keep the lungs inflated to the desired volume.

As will be understood by those skilled in the art, the frequency of administration may be determined by the route, the composition, and/or the purpose of administration. Following an initial administration, subsequent doses of the perfluorocarbon composition (the same composition and/or a different composition) may be administered (continuously or intermittently) to counteract perfluorocarbon gas losses and maintain the inflated lung volume or maintain the volume of perfluorocarbon gas in the lungs. Subsequent doses may be administered intravenously, through the airway (via inhalation), and/or through any other suitable route. Subsequent doses may also be calculated to either increase or decrease the steady state level of lung inflation, for example, in response to therapeutic or functional outputs, as described elsewhere herein, such that the therapy may be dynamic. The maintenance doses may be administered continuously (e.g., continuously infused) or as discrete doses spread out across discrete intervals of time. In some aspects, the maintenance dose is approximately 5%, 10%, 15%, 20%, or 25% that of the loading dose. In some aspects of the invention, PFC loading dose followed by continuous provision of maintenance doses (e.g., 5%, 10%, 15%, 20%, or 25% of the loading dose) can keep the alveoli from collapsing and maintain the lungs at a volume that improves lung compliance and/or decreases work of breathing for an extended period of time (e.g., during treatment of lung disease, e.g., ARDS) in lieu of application of PEEP.

Dosed administration includes, but is not limited to, hourly, four times a day, three times a day, two times a day, daily, once every other day, once every three days, once every four days, once every five days, once every 6 days, weekly, bi-weekly, monthly, bimonthly, quarterly, semiannually, annually, or any other suitable frequency. As will be appreciated by those skilled in the art, the disclosure is not so limited and includes any treatment or application for which alveolar delivery of the perfluorocarbon is suitable.

### V. Methods of Improving Ventilation

The present disclosure provides methods of improving ventilation in a subject. In some aspects, the subject is a human. In some aspects, the subject is on or in need of mechanical ventilation. In some aspects, the subject is not on and/or not in need of mechanical ventilation.

Without wishing to be bound by theory, in a clinical context many factors affect the decision to identify subjects or patients as “in need of mechanical ventilation” and/or to begin mechanical ventilation. Because no mode of mechanical ventilation can cure a disease process, the patient should generally have a correctable underlying problem that can potentially be resolved with the support of mechanical ventilation. In some aspects, mechanical ventilation is indicated when the patient's spontaneous ventilation is inadequate to sustain life, or as a measure to control ventilation in critically ill patients and as prophylaxis for impending collapse of other physiologic functions. Physiologic indications include respiratory or mechanical insufficiency and ineffective gas exchange. Without wishing to be bound by theory, common indications of the need of mechanical ventilation include, but are not limited to one or more of: bradypnea or apnea with respiratory arrest; ALI or ARDS; tachypnea (respiratory rate >30 breaths per minute); vital capacity less than 15 mL/kg; minute ventilation greater than 10 L/min; inadequate oxygenation, indicated by arterial partial pressure of oxygen (PaO₂) with a supplemental fraction of inspired oxygen (FIO₂) of less than 55 mm Hg; inadequate ventilation, indicated by arterial partial pressure of carbon dioxide (PaCO₂) greater than 50 mm Hg with an arterial pH less than 7.25; alveolar-arterial gradient of oxygen tension (A-a DO₂) with 100% oxygenation of greater than 450 mm Hg; clinical deterioration; respiratory muscle fatigue; obtundation or coma; hypotension; and neuromuscular disease. See Pham T et al., Mayo Clin Proc. 2017; 92(9):1382-1400 (doi: 10.1016/j.mayocp.2017.05.004), herein incorporated by reference in its entirety. In some aspects, the trend of the aforementioned factors and thresholds, and/or increasing severity of diseases, conditions, or symptoms described elsewhere herein influence clinical judgment as to whether subjects are in need of mechanical ventilation and/or when to initiate mechanical ventilation.

In some aspects of the present invention, provided herein are methods of inflating lungs of a subject by administering an effective amount of a composition comprising perfluorocarbon to the subject. In some aspects, the method inflates the subject's lungs by a predetermined volume. According to some aspects, the method inflates the subject's lungs by a volume approximately equal to 50%, 60%, 70%, 75%, 80%, 85%, 90%, 95%, or 100% of the lungs' residual volume (RV). According to some aspects, the method inflates the subject's lungs by a volume approximately equal to 50%, 60%, 70%, 75%, 80%, 85%, 90%, 95%, or 100% of the lung's functional residual capacity (FRC). According to some aspects, the method inflates the subject's lungs by a volume approximately no less than the RV and approximately no greater than the FRC. According to some aspects, the method inflates the subject's lungs by a volume that is approximately 5%, 10%, 15%, 20%, 25%, 30%, 35%, 40%, 45%, 50%, 55%, or 60% of the subject's total lung capacity (TLC). According to some aspects, the method inflates the subject's lungs by approximately 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9. 2.0, 2.1, 2.2, 2.3, 2.4, 2.5, 2.6, 2.7, 2.8, 2.9, or 3.0 L. According to some aspects, the method inflates the subject's lungs by a volume that is between about 0.1-0.5, 0.5-1.0, 1.0-1.5, 1.5-2.0, 2.0-2.5, 0.5-1.5, 0.5-2.0, 0.5-2.5, 1.0-2.0, 1.0-2.5, or 1.5-2.5 L. One or more loading doses may be delivered to a subject which are effective to quickly inflate the lungs by the predetermined volume (e.g., within 1, 2, 3, 4, 5, 6, 7, 8, 9, 10, 15, 20, 25, or 30 min).

In some aspects, provided herein are methods of reducing opening pressure of the lungs of a subject by administering an effective amount of a perfluorocarbon composition to the subject. According to certain aspects, the opening pressure of the lungs of the subject is reduced by at least about 5%, 10%, 15%, 20%, 25%, 30%, 40%, 50%, 60%, 70%, 80%, 90%, 95%, or 100%. According to some aspects, the opening pressure of the lungs of the subject is reduced by at least about 0.5 cm H₂O, 1 cm H₂O, 2 cm H₂O, 3 cm H₂O, 4 cm H₂O, 5 cm H₂O, 6 cm H₂O, 7 cm H₂O, 8 cm H2O, 9 cm H₂O, 10 cm H₂O, 11 cm H₂O, 12 cm H₂O, 13 cm H₂O, 14 cm H₂O, 15 cm H₂O, 16 cm H₂O, 17 cm H₂O, 18 cm H₂O, 19 cm H₂O, 20 cm H₂O, or more. The reduction in opening pressure may be based on a comparison to one or more measures or estimates of opening pressure in the subject prior to the treatment (e.g., a baseline) or to a control subject or population (e.g., having the same respiratory disease or condition). In some aspects, the opening pressure of the lungs of the subject is reduced to the level equal to or below the opening pressure of non-diseased lungs.

### VI. Methods of Treatment

The present disclosure provides methods of treating a subject experiencing a respiratory disease or condition or preventing a subject from developing a respiratory disease or condition, by administering an effective amount of a composition comprising perfluorocarbon (PFC) to the subject.

In some aspects, the subject is in pulmonary distress. In some aspects, the pulmonary disease or condition of the subject is one or more of: acute lung injury (ALI), acute respiratory distress syndrome (ARDS), neonatal respiratory distress syndrome, sudden acute respiratory syndrome (SARS), infectious lung disease, chemical pneumonitis, aspiration pneumonia, traumatic lung injury, pulmonary fibrosis, interstitial pneumonitis, and atelectasis. In some aspects, the subject has infection or infectious lung disease caused by one or more of SARS-CoV-2, SARS-associated coronavirus, influenza virus, parainfluenza virus, respiratory syncytial virus (RSV), Rhinovirus, Streptococcus pneumoniae, Klebsiella pneumoniae, Haemophilus influenzae, Bordetella pertussis, Chlamydia pneumoniae, Chlamydia psittaci, and Mycoplasma pneumoniae. In some aspects, the subject has ALI or ARDS caused by one or more of shock, trauma, sepsis, pneumonia, aspiration, burns, major surgery (e.g., surgery requiring cardiopulmonary bypass), blood transfusion, pancreatitis, or a severe viral infection, such as coronavirus infection like COVID-19 (i.e., SARS-CoV-2 infection).

ALI and its more severe form, ARDS, are injuries that result from a large-scale pulmonary inflammatory response. ALI and ARDS can be caused by, for example, inhalation of toxic gases, sepsis, pneumonia, trauma, shock, inflammatory storm, blood transfusion, pancreatitis, and aspiration. ALI and ARDS are characterized by an acute onset, pulmonary infiltrates, severe hypoxemia, hypercapnia, pulmonary edema, and substantial reduction in pulmonary compliance. Pronounced morphological changes occur in the lung parenchyma and are associated with impaired lung function. Common pathological characteristics of ALI and ARDS include an early exudative, inflammatory phase, followed in many patients by a fibrotic phase. (Ranieri V M et al., JAMA. 2012; 307:2526-33; Rubenfeld G D et al., N Engl J Med. 2005; 353:1685-93; Piantadosi C A & Schwartz D A, Ann Intern Med. 2004; 141:460-70; Bernard G R et al., Am J Respir Crit Care Med. 1994; 149:818-24; Ragaller M & Richter T, J Emerg Trauma Shock. 2010 January-March; 3(1): 43-51), each of which is incorporated by reference in its entirety. In ALI and ARDS, a decrease in surfactants lowers the lungs' ability to stretch and expand, causing reduced lung compliance, respiratory distress, and ultimately fibrosis. In addition, fluids leakage in the pulmonary parenchyma decreases O₂ diffusion, preventing organs from getting sufficient O₂.

A. Methods Associated with Mechanical Ventilation

Mechanical ventilation is often needed as supportive therapy for patients with ARDS or other forms of respiratory distress as part of the therapy for the underlying lung disease (e.g., inflammatory storms, shock, trauma, sepsis, pneumonia, aspiration, or burns) or needed when lungs are premature and lack a sufficient level of functional surfactant. However, mechanical ventilation commonly causes complications and further damages the lungs due to overinflation, increased positive pressure, overdistention of alveoli by high tidal volume, cyclic closing and reopening of the alveoli, inflammation, and O₂ toxicity (Ragaller M & Richter T, J Emerg Trauma Shock. 2010 January-March; 3(1): 43-51). In brief, under mechanical ventilation, the increased pressure required to inflate the collapsed alveoli (opening pressure) and/or high concentration of oxygen (fraction of inspired oxygen, FiO₂) in the gas mixture required to maintain adequate oxygenation and ventilation in the patient can induce or aggravate lung injury by disrupting lungs microbiome balance, causing additional inflammation, lung tears that typically result in pneumothorax and lung failure. (Cabrera-Benitez, N. E. et al., Anesthesiology, 2014. 121(1): p. 189), herein incorporated by reference in its entirety. This phenomenon, called ventilator-induced lung injury (VILI) or ventilator-associated lung injury (VALI), can further cause or trigger a pulmonary and systemic inflammatory reaction that may further lead to multiple organ dysfunction and multiple system organ failure. (Slutsky A S & Ranieri V M, N Engl J Med. 2013; 369:2126-36; Dreyfuss D & Saumon G, Am J Respir Crit Care Med. 1998; 157:294-323, each of which is incorporated by reference in its entirety).

Accordingly, in some aspects, disclosed herein are methods of treating a subject on mechanical ventilation or in need thereof by administering an effective amount of a perfluorocarbon composition to the subject. In some aspects, the subject is on mechanical ventilation at the time of the administration of the perfluorocarbon composition. In some aspects, the subject is dependent on continuous positive pressure mechanical ventilation, i.e., requiring PEEP. In some aspects, the opening pressure of the lungs of the subject is increased over normal/healthy baseline or control values prior to treatment. An opening pressure of diseased lungs, e.g., lungs of the subject on or in need of mechanical ventilation, may be elevated relative to that of non-diseased lungs. In some aspects, the methods disclosed herein reduce the opening pressure of the lungs of the subject by at least about 5%, 10%, 15%, 20%, 25%, 30%, 40%, 50%, 60%, 70%, 80%, 90%, 95%, or 100% compared to mechanical ventilation without administration of the perfluorocarbon composition as described herein. In some aspects, the methods disclosed herein reduce the opening pressure of the lungs of the subject by at least about 0.5 cm H₂O, 1 cm H₂O, 2 cm H₂O, 3 cm H₂O, 4 cm H₂O, 5 cm H₂O, 6 cm H₂O, 7 cm H₂O, 8 cm H2O, 9 cm H₂O, 10 cm H₂O, 11 cm H₂O, 12 cm H₂O, 13 cm H₂O, 14 cm H₂O, 15 cm H₂O, 16 cm H₂O, 17 cm H₂O, 18 cm H₂O, 19 cm H₂O, 20 cm H₂O, or more compared to mechanical ventilation without administration of the perfluorocarbon composition as described herein. The reduction in opening pressure may be based on a comparison to one or more measures or estimates of opening pressure in the subject prior to the treatment (e.g., a baseline) or to a control subject or population (e.g., having the same respiratory disease or condition). In some aspects, the method reduces the opening pressure of the lungs of the subject to the level equal to or below the opening pressure of non-diseased lungs.

In some aspects, the methods of the present disclosure may reduce an amount of PEEP needed for adequate oxygenation and ventilation in the subject. In some aspects, the amount of PEEP needed for adequate oxygenation and/or ventilation is reduced by at least about 5%, 10%, 15%, 20%, 25%, 30%, 40%, 50%, 60%, 70%, 80%, 90%, 95%, or 100% compared to mechanical ventilation without administration of the perfluorocarbon composition as described herein. In some aspects, the PEEP needed for adequate oxygenation and/or ventilation in a subject is reduced to at least about 25 cm H₂O, 24 cm H₂O, 23 cm H₂O, 22 cm H₂O, 21 cm H₂O, 20 cm H₂O, 19 cm H₂O, 18 cm H₂O, 17 cm H₂O, 16 cm H₂O, 15 cm H₂O, 14 cm H₂O, 13 cm H₂O, 12 cm H₂O, 11 cm H₂O, 10 cm H₂O, 9 cm H₂O, 8 cm H₂O, 7 cm H₂O, 6 cm H₂O, 5 cm H₂O, 4 cm H₂O, 3 cm H₂O, 2 cm H₂O, 1 cm H₂O, 0.5 cm H₂O, or less. In some aspects, the amount of PEEP needed is reduced to zero. In some aspects, the methods of the present disclosure may provide an increase in venous return as a result of a decrease in the amount of PEEP needed for adequate oxygenation and ventilation.

In some aspects, the methods of the present disclosure may reduce FiO₂ needed for adequate oxygenation in the subject. The subject may be on mechanical ventilation. In some aspects, the FiO₂ needed for adequate oxygenation is reduced by 0.05, 0.1, 0.2, 0.3, 0.4, 0.5, 0.6, 0.7, 0.75, or 0.79 compared to FiO₂ required without administration of the perfluorocarbon composition as described herein. In some aspects, the FiO₂ needed for adequate oxygenation is reduced to 0.95, 0.9, 0.8, 0.7, 0.6, 0.5, 0.45, 0.4, 0.35, 0.3, 0.25, or 0.21 (room air).

In some aspects, the methods of the present disclosure may mitigate the extent of ventilator-induced lung injury (VILI) or ventilator-associated lung injury (VALI) in a subject. For instance, the extent of one or more of barotrauma (due to increased positive pressure), volutrauma (due to over-distention of alveoli by high tidal volume), pneumothorax, mechanical shear stress (due to cyclic closing and reopening of alveoli), biotrauma and inflammation (due to inflammatory mediators released by shear stress), pulmonary fibrosis, and/or O₂-toxicity (due to high FiO₂ and generation of toxic oxygen radicals) may be mitigated in a subject. In some aspects, the degree of mitigation is by at least about 5%, 10%, 15%, 20%, 25%, 30%, 40%, 50%, 60%, 70%, 80%, 90%, 95%, or 100%. In some aspects, the methods of the present disclosure may prevent the subject from developing VILI or VALI.

In some aspects, the method of the present disclosure decreases the length of time the subject is placed on mechanical ventilation or prevents the subject from requiring mechanical ventilation. In some aspects, the method of the present disclosure decreases the length of time the subject is placed on mechanical ventilation by at least about 5%, 10%, 15%, 20%, 25%, 30%, 40%, 50%, 60%, 70%, 80%, 90%, 95%, or 100% compared to mechanical ventilation without administration of the perfluorocarbon composition as described herein.

In some aspects, the subject is on mechanical ventilation at the time of the administration of the perfluorocarbon composition, and the subject is weaned from mechanical ventilation after the administration of the perfluorocarbon composition. “Weaning” from mechanical ventilation as used herein refers to discontinuation from mechanical ventilation, for instance by extubation, and transition to spontaneous breathing. By way of example, weaning may be considered in a clinical context when the event that precipitated the patient's need for mechanical support is adequately addressed. Patients may be evaluated each day to determine if they are a candidate for weaning. Patients who may be able to support their own ventilation and oxygenation, and thus candidates for weaning, may be identified by considering each patient's clinical scenario and various factors including, but not limited to: improvement or resolution of the process responsible for the patient's respiratory failure, for instance respiratory diseases and conditions disclosed elsewhere in the present disclosure; hemodynamic stability of the patient; absence of cardiac complications, such as active ischemia, unstable arrhythmias, and vasopressor support; adequate oxygenation (e.g., PaO₂ of greater than 60 mm Hg with an FiO₂ of less than 40% and a PEEP of less than 5 cm H₂O); appropriate mental and neuromuscular statuses on minimal or no sedation; adequate strength of the respiratory muscles; appropriate acid-base status and electrolyte status; and appropriate systemic conditions, including no fever and normal adrenal and thyroid functions. See Pham T et al., Mayo Clin Proc. 2017; 92(9):1382-1400 (doi: 10.1016/j.mayocp.2017.05.004); Rose L et al., Intensive Crit Care Nurs. 2015; 31(4):189-95 (doi: 10.1016/j.iccn.2015.07.003), each of which is herein incorporated by reference in its entirety. In some aspects, the method of the present disclosure causes weaning from mechanical ventilation of a subject at an earlier time point compared to mechanical ventilation without administration of the perfluorocarbon composition as described herein. For example, the methods of the present disclosure may cause an earlier weaning of a subject from mechanical ventilation by at least about 12 hours, 1 day, 2 days, 3 days, 4 days, 5 days, 6 days, 7 days, two weeks, three weeks, four weeks, one month, two months, or longer compared to mechanical ventilation without administration of the perfluorocarbon composition as described herein.

In some aspects, the subject is at risk of requiring mechanical ventilation at the time of the administration of the perfluorocarbon composition, wherein the administration of the perfluorocarbon composition according to the disclosed method prevents the subject from requiring mechanical ventilation or prevents the subject from requiring mechanical ventilation at as high a PEEP compared to mechanical ventilation without administration of the perfluorocarbon composition as described herein. In some aspects, the placing of a subject on mechanical ventilation is effectively delayed (e.g., as an underlying condition continues to develop). For example, the subject may not be placed on mechanical ventilation for an additional 1, 2, 3, 4, 5, 6, 7, 8, 9, 10, 11, 12, 13, or 14 days, or 2, 3, 4, 5, or more weeks relative to the time the subject would be placed on mechanical ventilation without administration of the perfluorocarbon composition as described herein. In some aspects, the subject is placed on mechanical ventilation at a lower PEEP compared to the PEEP the subject would require without administration of the perfluorocarbon composition as described herein. For example, the opening pressure of the lungs of the subject may be reduced by at least about 5%, 10%, 15%, 20%, 25%, 30%, 40%, 50%, 60%, 70%, 80%, 90%, 95%, or 100% compared to mechanical ventilation without administration of the perfluorocarbon composition as described herein. In some aspects, the methods disclosed herein reduce the opening pressure of the lungs of the subject by at least about 0.5 cm H₂O, 1 cm H₂O, 2 cm H₂O, 3 cm H₂O, 4 cm H₂O, 5 cm H₂O, 6 cm H₂O, 7 cm H₂O, 8 cm H₂O, 9 cm H₂O, 10 cm H₂O, 11 cm H₂O, 12 cm H₂O, 13 cm H₂O, 14 cm H₂O, 15 cm H₂O, 16 cm H₂O, 17 cm H₂O, 18 cm H₂O, 19 cm H₂O, 20 cm H₂O, or more compared to mechanical ventilation without administration of the perfluorocarbon composition as described herein. In some aspects, the method reduces the opening pressure of the lungs of the subject to the level equal to or below the opening pressure of non-diseased lungs.

### B. Therapeutic Effects

The present disclosure further provides methods of improving the respiratory status or function of a subject, or preventing decline or deterioration of the respiratory status or function, by administering an effective amount of a perfluorocarbon composition to the subject. In some aspects, the subject has decreased, impaired, or suboptimal respiratory status or function. For instance, the subject has one or more of hypoxemia (decreased O₂ concentration in the blood), hypoxia (decreased O₂ concentration in the tissue), hypercapnia (increased CO₂ concentration in the blood), reduced lung compliance, reduced forced vital capacity (FVC), and reduced diffusing capacity (DLCO). In some aspects, the subject's respiratory status or function is within normal range, but the subject is at risk of developing decline of the respiratory status or function. Accordingly, in some aspects, the methods of the present disclosure improve one or more of the parameters of respiratory status in the subject.

In some aspects, the method comprises administering an effective amount of a perfluorocarbon composition to the subject, wherein the subject has surfactant deficiency or dysfunction, and wherein the method improves surfactant deficiency and dysfunction in the subject. In some aspects, the methods of the present disclosure result in an increased compliance of the alveoli or the lungs of the subject. In some aspects, the methods may increase the compliance by at least about 5%, 10%, 15%, 20%, 25%, 30%, 40%, 50%, 60%, 70%, 80%, 90%, 100%, 200%, 300%, 400%, 500%, 600%, 700%, 800%, 900%, 1000%, or more. In some aspects, the methods increase the compliance of the alveoli or the lungs of the subject to the level of non-diseased alveoli or lungs. By way of example, a normal human may have a lung compliance between about 100 and 400 mL/cm H₂O (e.g. 200 mL/cm H₂O). By further way of example, a subject having ARDS and/or a subject having reduced lung compliance may have a compliance no greater than about 90, 80, 70, 60, 50, 40, 30, 20, or 10 mL/cm H₂O).

In some aspects, the methods of the present disclosure cause inflation of the lungs of the subject. In some aspects, the methods of the present disclosure allow maintenance of a higher lung volume at the end of expiration, e.g., a higher functional residual capacity (FRC) or a higher residual volume (RV).

In some aspects, the methods of the present disclosure reduce, decrease, diminish, or prevent collapse of alveoli at the end of exhalation in the subject. In some aspects, the methods of the present disclosure reduce opening pressure of the subject's lungs. In some aspects, the methods of the present disclosure reduce, decrease, diminish, or prevent atelectasis.

In some aspects, the methods of the present disclosure reduce or prevent inflammation in the lung of the subject. In some aspects, the methods of the present disclosure reduce, diminish, or prevent pulmonary edema in the subject. Without wishing to be bound by theory, the reduction or prevention of inflammation may result directly from an anti-inflammatory effect of perfluorocarbon compositions (see Kacmarek R M et al., Am. J. Respir. Crit. Care Med., 2006, 173, 882-89; Kawamae K et al., Crit Care Med 2000; 28:479-483; Thomassen M J et al., Crit Care Med 1997; 25:2045-2047, each of which is herein incorporated by reference in its entirety) and/or indirectly for instance from one or more of: reduction of PEEP or FiO₂ needed for adequate oxygenation and/or ventilation, improvement of respiratory status, reduction or prevention of VILI or VALI, and improvement of underlying respiratory conditions, e.g., ALI, ARDS, and reduction or prevention of associated inflammation.

In some aspects, the methods of the present disclosure enhance or normalize oxygen delivery and/or gas exchange in the alveoli of the subject. In some aspects, the methods of the present disclosure improve ventilation-perfusion mismatch (V-Q mismatch) in the lungs of the subject. In some aspects, the methods of the present disclosure improve diffusing capacity (DLCO) in the lungs of the subject.

In some aspects, the methods of the present disclosure cause improvement or normalization of one or more of arterial or venous (blood) O₂ concentration, arterial or venous (blood) CO₂ concentration, arterial or venous (blood) pH, and tissue O₂ concentration.

In some aspects, the methods of the present disclosure cause improvement or normalization of blood oxygen saturation as detected, for example, by a pulse oximeter or by blood gas analysis.

In some aspects, the methods of the present disclosure reduce or diminish the length of time the subject is on mechanical ventilation, or prevent the subject from requiring mechanical ventilation. In some aspects, the method of the present disclosure reduce the PEEP used with mechanical ventilation, as described elsewhere herein, or reduce or diminish the length of time the PEEP is maintained at a certain level.

In some aspects, the methods of the present disclosure reduce or diminish the length of time the subject is on supplemental oxygen, or prevent the subject from requiring supplemental oxygen. In some aspects, the method of the present disclosure reduces the FiO₂ to be provided by supplemental oxygen for adequate oxygenation in the subject. Higher levels of FiO₂ in supplemental oxygen are known to be more injurious to the lungs. For example, according to some aspects, an FiO₂ of at least about 0.6 (supplemental oxygen of 60% inhaled gas) may be presumed to be injurious. According to some aspects, the methods described herein may reduce or diminish the length of time the subject is on supplemental oxygen having an FiO₂ of at least about 0.6, or prevent the subject from requiring supplemental oxygen having an FiO₂ of at least about 0.6. According to some aspects, the methods described herein may reduce the FiO₂ of supplemental oxygen being provided or to be provided to a subject to a level below about 0.6. Other suitable thresholds are known in the art and may depend on the judgement of the clinician (e.g., the FiO₂ threshold may be approximately 0.50, 0.55, 0.65, 0.70, 0.75, 0.80, etc.).

In some aspects, the methods of the present disclosure reduce, diminish, or prevent one or more symptoms associated with the respiratory disease or condition (e.g. respiratory distress) in the subject, including but not limited to any of the symptoms described elsewhere herein. In some aspects, the one or more symptoms are one or more of dyspnea, wheezes, chest tightness, and cough. In some aspects, the methods of the present disclosure improve exercise tolerance in the subject.

In some aspects, the methods of the present disclosure reduce or diminish the length of time the subject is hospitalized or admitted to ICU, or prevents the subject from being hospitalized or admitted to ICU. In some aspects, the methods of the present disclosure reduce or diminish the frequency of times the subject is hospitalized or admitted to ICU. In some aspects, the methods of the present disclosure cause faster recovery of the respiratory disease or condition, or underlying disease or condition in the subject. In some aspects, the methods prevent the subject from developing a respiratory disease or condition.

### C. Improving Gas Transfer and Inhalation Drug Delivery

As discussed elsewhere herein, the methods of present disclosure may improve gas transfer in a subject. In some aspects, the subject has atelectasis and/or a ventilation-perfusion mismatch (V-Q mismatch), or is at risk of developing atelectasis and/or a V-Q mismatch. According to certain aspects, the methods of the present disclosure improve gas transfer and/or improve or prevent the atelectasis or the V-Q mismatch. In some aspects, the atelectasis was caused by one or more of undergoing anesthesia, prolonged bed rest with few changes in position, shallow breathing, underlying lung disease (e.g., tumor obstructing the airway, pneumonia), and obstruction of the airway by a foreign object. Accordingly, in some aspects, the subject who would benefit from administration of the perfluorocarbon composition is a person undergoing or in need of prolonged bed rest, e.g., a person with a chronic or significant disease; a person in need of prolonged sedation, e.g., a patient under or recovering from anesthesia, a patient undergoing or recovering from surgery, a psychiatric patient, an ICU patient, or a critically ill patient; or a subject with restrictive lung disease, e.g., pulmonary fibrosis, interstitial pneumonitis, asbestosis, occupational lung disease, sarcoidosis). In some aspects, the subject who would benefit from administration of the perfluorocarbon composition is one or more of the following, to the extent administration is not contraindicated by an increased RV, FRC, or TLC: a subject with an underlying condition that cases decreased tidal volume, e.g., a subject experiencing pain; a patient recovering from surgery; a subject with underlying respiratory condition; a subject with decreased cough reflex, e.g., an elderly subject, a subject with weakened or paralyzed respiratory muscles. In some aspects, the subject is not on and/or is not in need of mechanical ventilation. In some aspects, the subject is on or is in need of mechanical ventilation. In some aspects, the subject has a respiratory disease or condition. In some aspects, the subject is at risk of developing a respiratory disease or condition

In further aspects, the methods of the present disclosure cause an increased efficacy of delivery of therapeutic agents by inhalation. Inhalation therapeutic agents may include, but are not limited to, a bronchodilator, an antibiotic, an antimicrobial agent, an anti-inflammatory agent, an anti-cancer agent, a steroid, an immunosuppressive agent, an immune modulating agent, a pulmonary surfactant, a therapeutic peptide (e.g., an antibody), a therapeutic nucleotide (e.g., plasmid, DNA, antisense RNA, siRNA, shRNA). In some aspects, the perfluorocarbon composition of the present disclosure and an inhalation therapeutic agent may be administered at the same time and/or in the same combination, e.g., by inhalation, or the additional therapeutic agent/modality can be administered as part of a separate composition or at separate times or by a separate dosing regimen and/or by another method known in the art or described herein. In some aspects, the subject has an underlying respiratory disease or condition. In one aspect, the respiratory disease or condition is one or more of pulmonary fibrosis, interstitial pneumonitis, infectious lung disease, chemical pneumonitis, asthma, and lung cancer. In some aspects, the subject is not on and/or is not in need of mechanical ventilation. In some aspects, the subject is on or is in need of mechanical ventilation.

### D. Coadministration

The invention further provides methods and uses of a perfluorocarbon composition for improving the respiratory status or function in a subject, in combination with other pharmaceuticals and/or other therapeutic methods, e.g., with known pharmaceuticals and/or known therapeutic methods, such as, for example, those which are currently employed for treating the diseases or conditions of the subject, e.g., ARDS and its underlying conditions.

Accordingly, in some aspects of the invention, the methods of administering an effective amount of a perfluorocarbon composition to the subject further include administering to the subject one or more additional therapeutic agents and/or therapeutic modalities. The perfluorocarbon composition and an additional therapeutic agent and/or modality may be administered at the same time and/or in the same combination, e.g., intravenously, or the additional therapeutic agent/modality can be administered as part of a separate composition or at separate times and/or by another method known in the art or described herein.

In some aspects, the subject is experiencing ALI or ARDS, and the additional therapeutic agent or therapeutic modality is one or more of a steroid, an antibiotic, a diuretic, a vasopressor, a surfactant, fluid resuscitation, and bronchoscopy.

According to some aspects, the perfluorocarbon composition (e.g., a nanodroplet emulsion or microbubble dispersion) may be used to deliver a therapeutic to lungs (e.g., the alveoli). The therapeutic may comprise a biomolecule (e.g., a protein or nucleic acid) or synthetic molecule. The therapeutic may be loaded into the core of a perfluorocarbon particle (enclosed within the surfactant shell), embedded into the surfactant shell of a perfluorocarbon particle, and/or covalently or non-covalently coupled with the surfactant shell (on its internal and/or external surface). The therapeutic may be configured to diffuse across the capillary endothelial layer and, depending on the target, the alveolar cell as well as the interstitial space between them. The evaporation of the perfluorocarbon into the alveoli may be configured to release the therapeutic into the interstitial space and/or the alveoli. The therapeutic may comprise a targeting molecule (e.g., antibody or cell receptor ligand) for targeting a particular relevant cell type. The therapeutic may be configured to treat the underlying respiratory disease or condition for which the perfluorocarbon composition is administered, as described elsewhere herein, and/or may be used to treat an associated condition. Such methods may provide improved supportive care in addition to therapy for a respiratory disease or condition. Suitable agents and methods for drug delivery via perfluorocarbon particles are known in the art.

### EXAMPLES

### Example 1. PFB and PFP Nanodroplet Production

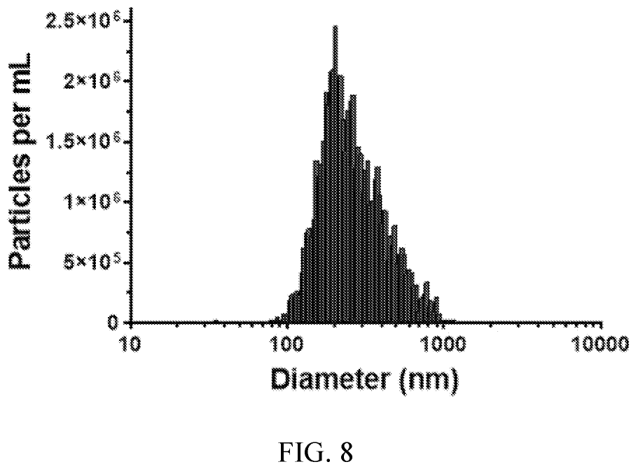

Perfluorobutane (PFB) gas was allowed to condense inside a 1 mL syringe inclined and covered in dry ice with the syringe neck pointing upward. 2 mL of an 80:15:5 (v:v:v) PBS/propylene glycol/glycerol excipient solution was added to a first dram vial containing a dry phospholipid film. The phospholipid film comprised 1,2-distearoy 1-sn-glycero-3-phosphocholine DSPC and 1,2-distearoyl-sn-glycero-3-phosphoethanolamine-N-methoxy(polyethyleneglycol)-2000 (DSPE-PEG2000) in a 9:1 molar ratio. Other suitable phospholipid compositions and methods for producing the phospholipid film are well known in the art. The first dram vial was then heated on a 70° C. heating block for 15 min, followed by sonication in a bath sonicator until the solution turned clear. The phospholipid solution was allowed to cool to room temperature and then cooled in an ice-salt bath (between −10 and −12° C.) for 5 minutes. Another ice-salt bath (between −10 and −12° C.) was prepared and a second dram vial was placed inside for receiving the liquid PFB and cold phospholipid mixture such that the bath covers the entire height of the dram vial. 150 μL liquid PFB was transferred to the second dram vial and the ice-salt bath was moved under a probe sonicator. The phospholipid solution was gently transferred into the second dram vial. The phospholipid and PFB mixture was sonicated with the probe sonicator for 10 s at 20% power. If a drop of non-encapsulated PFB remained at the bottom of the vial, the mixture was sonicated once more. The resulting emulsion was transferred to a 3 mL syringe and centrifuged at 200×g for 1 min, making sure to fill the cap with excipient solution. After centrifugation, the emulsion was transferred to a vial for storage at 4° C. By way of example, a concentration of 70 μL PFB/mL (7% v/v) produces approximately 10¹² 100-500 nm PEGylated droplets per mL, as seen in the size distribution of FIG. 8. PFB nanodroplets used in all subsequent experiments were produced accordingly. Concentrations, however, can be of any range that allows the emulsion to remain stable, including those described elsewhere herein. In an alternative method, PFB is condensed to a liquid and emulsified with phospholipids at low temperature, as described in US 2018/0272012.

Perfluoropropane (PFP) nanodroplets may be prepared using the above procedure with minor modifications. The ice baths are prepared as ethanol/dry ice (around −70° C.) and the excipient solution is 4:6 (v:v) PBS/propylene glycol.

The total gas volume generated by vaporizing the liquid emulsions prepared as described above was quantified by gas chromatography-mass spectrometry (GC-MS) for comparison to theoretical values. The results are illustrated in Table 1 below in which row (a) depicts the experimental volume used for the emulsion formulation, row (b) depicts the experimental gas volume observed in a 3 mL syringe post vaporization, and row (c) depicts the experimental gas volume and encapsulation efficiency (EE) quantified by GC-MS analysis (m/z 69 and 119, n=7). A calibration curve for a set of PFB in air standards (0.04-1% v/v) was linear between gas concentration and area under the peak.

TABLE 1
Theoretical and Experimental Gas Volumes
Generated from PFB Nanodroplet Emulsions

| | | Liquid | Theoretical | Experimental | |
|---|---|---|---|---|---|
| | Emulsion | PFB | PFB Gas | PFB Gas | |
| | Volume | Volume | Volume | Volume | % |
| Row | (mL) | (μL) | (mL) | (mL) | EE |
| (a) | 2.15 | 150 | 22.95 | | |
| (b) | 0.1 | 7 | 1.1 | 1-1.1 | |
| (c) | 0.01 | 0.7 | 0.11 | 0.113 ± 0.03 | 98 ± 22 |

### Example 2. Determination of PFB Nanodroplet Emulsion Loading Dose Required to Inflate Normal Lungs

Tracheostomy was performed on six 180-200 g normal rats anesthetized with ketamine/xelazine (K/Z) (having estimated vital capacities of about 9 mL). The rats were intravenously infused with a 7% (v/v) PFB nanodroplet emulsion at 150 μL/min—two infused with 110 μL of the PFB nanodroplet emulsion (theoretically predicted to generate 1.2 mL of gas), two infused with 230 μL of the PFB nanodroplet emulsion (theoretically predicted to generate 2.4 mL of gas) and two infused with 450 μL of the PFB nanodroplet emulsion (theoretically predicted to generate 4.8 mL of gas). One minute after the end of the infusion, rats were euthanized and the volume of air that filled the lungs with the chest intact at 35-cm water hydrostatic pressure was measured.

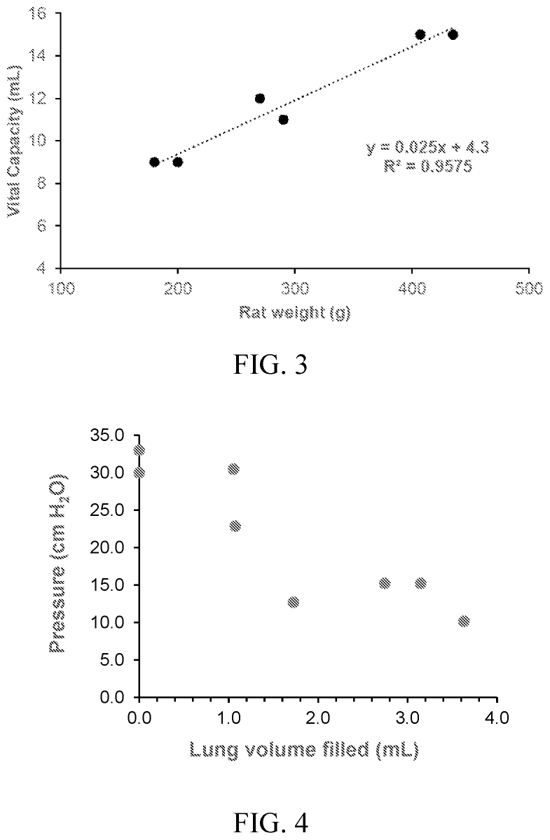

Similar volumes can be calculated using a pressure sensor (vital capacity=[(P₁−P₂)/2]Πr²; where P₁ and P₂ are the pressure measured by an Arduino pressure sensor before and after opening the water column to the trachea, r is the radius of the chamber in cm, Π is pi). The measured volume was subtracted from the theoretical volume measured in control rats (vital capacity, FIG. 3, vital capacity (mL)=0.025 rat weight (g)+4.3; R²˜0.96) to calculate the volume of the lungs filled by PFB gas. Lung volumes filled by PFB gas are shown in FIG. 2, very closely aligning to the theoretical calculations.

The slope of the correlation line in FIG. 2 indicates that 1 mL of emulsion containing 70 μL of PFB liquid inflated the lungs by 9.42 mL instead of the predicted 10.6 mL. This discrepancy was likely due, in part, to PFB gas exhalation during the 1-min prior to animal sacrifice and a small amount of PFB liquid that had not yet vaporized, suggesting that PFB vaporizes immediately when it reaches alveoli. When an emulsion of the less volatile DDFP (BP=28° C.) was infused that should theoretically inflate the lungs by 2.4 mL, lungs were not inflated 1 min post infusion, indicating that liquid DDFP does not vaporize into the lungs as rapidly as PFB despite its known relatively rapid exhalation, as reported in Correas et al., Ultrasound Med Biol. 2001 April; 27(4):565-70 (doi: 10.1016/s0301-5629(00)00363-x), which is herein incorporated by reference in its entirety.

### Example 3. Determination of PFB Nanodroplet Emulsion Loading Dose Required Reduce Opening Pressure

Immediately after the completion of the experiments described in example 2, the lungs were then lavaged 3 times with saline to decrease the surfactant levels and reduce the lung compliance, and the opening pressure was measured by recording the height of a water column required, relative to the rat's chest, that began moving air into the lungs. As shown in FIG. 4, the opening pressure decreased by about 50% when the lungs were inflated with approximately 2 mL of PFB gas (e.g., via infusion with the 230 μL emulsion), which is approximately the functional residual capacity (FRC) in the approximately 200 g rats. There was little change thereafter for increased volumes of the PFB nanodroplet emulsion, suggesting that additional inflation was not necessary.

### Example 4. Comparison of PFB/DDFP and DDFP Nanodroplet Emulsions on Respiratory Frequency and Lung Inflation

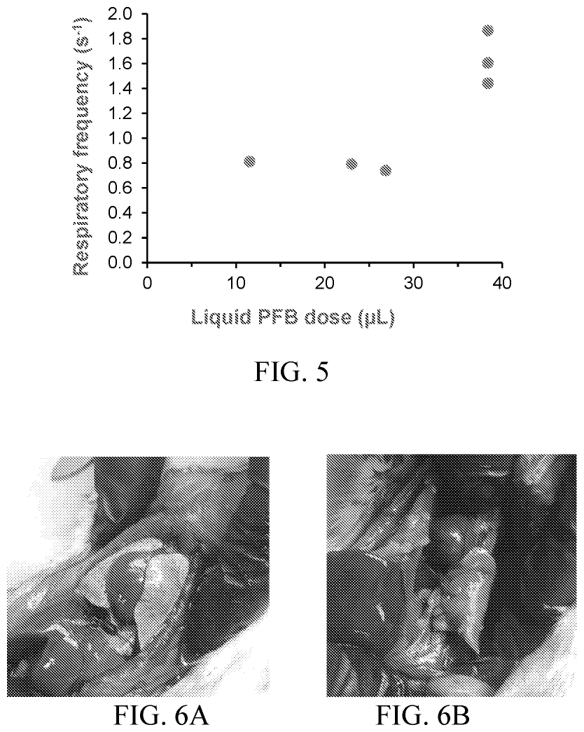

Two normal 600 g rats were gradually intravenously infused with 1.1 mL of a 7% (v/v) PFC nanodroplet emulsion over a 10 min period—one with a PFB/DDFP (1:1) nanodroplet emulsion (theoretically predicted to generate a maximum volume of approximately 6 mL of PFB gas and 5 mL of DDFP gas) and one with a 1.1 mL DDFP nanodroplet emulsion (theoretically predicted to generate a maximum volume of approximately 10 mL of DDFP gas). The respiratory frequency was monitored by ultrasound upon injection of the emulsion and over the 10 minute period. No substantial changes to respiratory frequency was observed for the DDFP emulsion over the period. As shown in FIG. 5, no substantial changes in respiratory frequency were observed for the PFB/DDFP (1:1) nanodroplet emulsion until after approximately 30 μL of PFB liquid was injected. The rats were euthanized and the chest cavity opened. As shown in FIGS. 6A and 6B, only the rat infused with the PFB/DDFP (1:1) emulsion (FIG. 6A) and not the rat infused with the DDFP emulsion (FIG. 6B) displayed inflated lungs. Ultrasound also confirmed that the nanodroplets were thermally stable and did not phase change in vivo during circulation.

### Example 5. Determination of PFB Nanodroplet Emulsion Loading Dose Required to Inflate Injured Lungs to Normal RV or FRC

Normal lungs and bleomycin-injured lungs are studied to define the loading dose of PFB nanodroplet emulsion needed to fill normal and injured lungs to FRC. Briefly, to induce bleomycin injury (mimicking ARDS), rats are anesthetized under ketamine/xylazine (K/Z) and bleomycin (8 U/kg, in 250 μl clinical grade saline) is instilled in the trachea (IT). Bleomycin induces an inflammatory response, recruits inflammatory cells, increases chemokine release and causes surfactant dysfunction to mimic ARDS, as reported in Savani et al., Am J Physiol Lung Cell Mol Physiol. 2001 September; 281(3):L685-96 (doi: 10.1152/ajplung.2001.281.3.L685), which is herein incorporated by reference in its entirety. At 7 days after bleomycin injury, at which time inflammation peaks, all rats are anesthetized with 0.2 mL of a 10:1 ketamine/xylazine cocktail given via intraperitoneal injection. Following tracheostomy, rats are attached via a tracheotomy tube to the FLEXIVENT® ventilator (SCIREQ®), the gold standard for measuring lung function parameters in rodents. The FLEXIVENT® is an integrated platform that combines a computer-controlled piston ventilator with advanced lung modelling capabilities for a comprehensive assessment of lung function. It attaches to a tracheostomy tube and ventilates the rat to acquire not only fractional lung volumes, but also generates pressure-volume loops to assess compliance and opening pressure. Specific features that are unique to the FLEXIVENT® are as follows: Lung conditioning maneuvers and standardization of breathing history, estimates of inspiratory capacity, dynamic resistance and compliance measurements, pressure-volume loop, resistance of conducting airway vs. peripheral airways and tissue compliance/elastance, static compliance using pressure-volume loop, automated dose-response curves for airway hyper-responsiveness studies and lung volumes. Using fully controlled experimental conditions, use of FLEXIVENT® ensures reproducible outcomes and allow testing that pre-filling the lung with PFC gas given as a liquid emulsion intravenously (IV) 1) improves lung compliance, 2) decreases opening pressure, 3) decreases or eliminates PEEP pressure and 4) improves gas exchange. Fractional lung volumes and compliance are measured continuously from baseline and up to 5 min after infusion of saline or up to 0.1, 0.2, 0.3, or 0.4 mL of infusing PFB nanodroplet emulsion when peak filling of the lungs is expected to occur. A correlation line defining peak residual lung volume relative to PFB nanodroplet emulsion dose is determined to define the PFB gas volume generated per mL of emulsion. Rats with normal lungs injected with saline are used to define the normal FRC and RV, which in turn defines the loading PFB dose required to fill the FRC/RV via the correlation line. All rats are euthanized and the degree of lung injury is assessed histologically by a veterinary pathologist to ensure that a similar degree of injury occurred in all bleomycin-treated lungs.

### Example 6. Determination of PFB Nanodroplet Emulsion Infusion Rate Required to Keep Injured Lungs Inflated at Normal FRC

Normal and bleomycin-injured lungs are studied using a plethysmograph with a head-out configuration to define the maintenance infusion rate needed to keep the lungs inflated at FRC in conscious rats. Bleomycin injury is induced as described above. All rats receive an indwelling IV line placed in the jugular vein, advanced into the superior vena cava, secured, and exteriorized through the back. At 7 days after bleomycin injury, at which time inflammation peaks, the plethysmography experiment is conducted. The IV line is filled with heparin 3 days before the plethysmography experiment. To reduce stress-related respiratory effects of the rats being placed in the plethysmograph chamber, the rats are acclimated to the chamber for 1-2 hours several times the week before the experiment and for 30 min prior to collecting data. The IV line is exposed and exteriorized from the chamber 30 min before the experiment. Once baseline values stabilize, the loading PFB dose determined in Example 5 is infused through the IV line without disturbing the animal. The respiratory rate is monitored from before infusion to 1 hour after infusion. Gas samples are collected for characterization of PFB content by Gas Chromatography from the outflow before infusion and at least about every 20 sec, or 30 sec, or 45 sec, or 1 min for at least about 6 min, as well as every 6 min after the infusion up to an hour after infusion. The infusion rate of PFB nanodroplet emulsion (e.g., mL/min) to maintain the lungs inflated to FRC is determined to be the percent of PFB gas lost per minute after peak inflation converted to mL emulsion using the correlation line define in Example 5. All rats are euthanized and the degree of lung injury is assessed histologically by a veterinary pathologist to ensure that a similar degree of injury occurred in all bleomycin-treated lungs.

### Example 7. Determination of PFB Nanodroplet Emulsion Loading Dose and PEEP Required to Normalize Blood Gases in Subjects with Injured Lungs

Normal lungs and bleomycin-induced injured lungs are anesthetized with 0.2 mL of a 10:1 ketamine/xylazine cocktail given via intraperitoneal injection. The rats are instrumented with a catheter in the superior vena cava for blood sampling to measure pH; a pulse oximeter on the front paw to monitor O₂ saturation; an OXYLITE™ Pro sensor (OXFORD OPTRONIX LTD.) in the aorta to monitor arterial pO₂; and a tracheostomy tube connected to a FLEXIVENT® ventilator (SCIREQ®) to measure fractional lung volumes, compliance and opening pressure. Once baseline values stabilize, a PFB nanodroplet emulsion loading dose determined in Example 5 will be infused followed by a slow infusion at the rate determined in Example 6 to maintain the lungs inflated to FRC. All parameters are continuously monitored for all measurements except pH, which is assessed at baseline and at 5, 10, 20, 40 and 60 minutes after the administering the loading dose. The rats are monitored for endpoints comprising the normalization of all lung and blood parameters of rats with injured lungs and no deterioration of these parameters in rats with normal lungs. Initially rats are ventilated with no PEEP (supported by the FLEXIVENT® ventilator) to determine if maintaining lungs inflated with PFC gas at FRC will decrease opening pressure needed to ease ventilation. If blood gases do not normalize in rats with injured lungs, the experiment is repeated with additional rats having PEEP ventilation incremented by 3 cm of water. The PEEP is continually increased by 3 cm of water in additional experiments of six additional rats until blood gases normalize to determine whether PEEP is necessary after PFC gas filling. Similarly, if blood gases normalize with no PEEP, the experiment is repeated with additional rats in which the PFB nanodroplet emulsion loading dose is decreased by 20%. The loading dose is continually decreased by 20% in additional experiments of six additional rats until blood gases do not normalize to determine the minimal PFB dose necessary to normalize blood gases.

### Example 8. Effect of Infused Nanodroplets on Inspiratory Capacity and Static Compliance

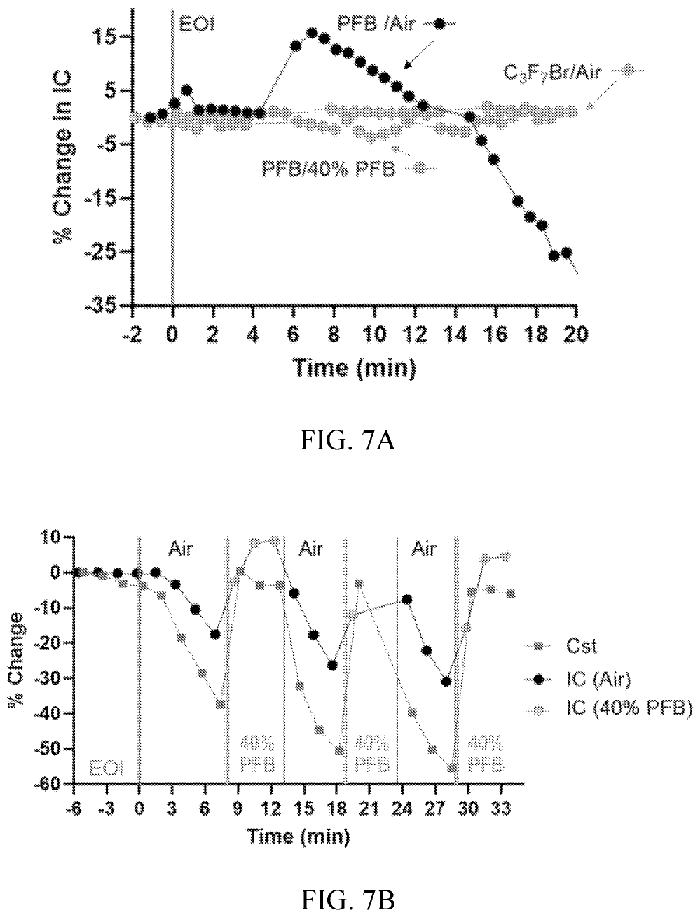

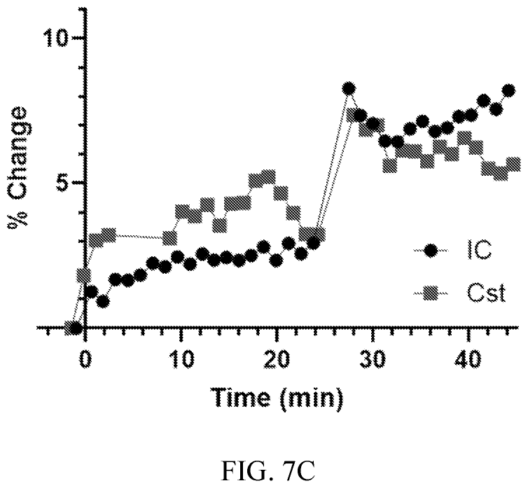

Three normal rats were anesthetized with ketamine/xyalazine (K/Z), placed on a tracheostomy tube, and connected to a FLEXIVENT® ventilator as described in Example 5. At the end of normal expiration, the inspiratory capacity (IC) was measured with the ventilator by measuring the volume required to inflate the lungs to reach 32 cm H₂O. Following baseline measurements, a 100 μL nanodroplet emulsion having 7 μL of either C₃BrF₇ (n=1) (BP=12° C.) or PFB (n=2) was intravenously infused over a period of 40 s. The rats breathed either air or a gas mixture containing 40% PFB, 8% O2 and 52% air, as indicated in FIG. 7A. FIG. 7A shows the percent change in inspiratory capacity over 20 minutes following the infusion. As can be seen in FIG. 7A in the rat infused with PFB during air breathing, inspiratory capacity increased slightly and then decreased by 25% (2.1 mL) by 20 min, essentially doubling the expected 1.1 mL PFB gas volume generated. The infusion of C₃BrF₇ during air breathing and PFB during 40% PFB breathing had no effect on inspiratory capacity. As to C₃BrF₇, the bromine itself may increase lipid solubility, but a slower vaporization rate might also explain the difference.

To confirm the impact of adding PFB vapor to the inhaled gas, the experiment was repeated with a fourth normal rat infused with the PFB nanodroplet emulsion, but alternating the inhaled gas between air and gas comprising 60% air and 40% PFB. Inspiratory capacity was measured along with static compliance calculated from pressure-volume (PV) loops, as shown in FIG. 7B. In all cycles, inspiratory capacity and compliance decreased during air breathing, but recovered within 1 min when switched to 40% PFB breathing. No foam exudates were observed from freshly cut lung surfaces in these rats. These results confirm that the 1 mL of PFB gas in alveoli caused an additional 1 mL of air to be trapped in the lung after 20 min, known as “air trapping.” The rapid recovery of inspiratory capacity and compliance observed during inhalation of gas having PFB vapor may be explained in part by gas osmosis, as described in Schutt et al., Artif Cells Blood Substit Immobil Biotechnol. 1994; 22(4):1205-14 (doi: 10.3109/10731199409138817), which is herein incorporated by reference in its entirety, but the observed speed of recovery suggests that other factors likely exist as well. These results, therefore, demonstrate that lung air trapping occurs in rats shortly after infusing non-brominated low boiling point PFCs and that the air trapping can be rapidly reversed by adding PFB to the inspired gas. To date, air-trapping has not been reported in human. This is likely because of the large number of lung macrophages in some species that create a PFC pool adjacent to alveoli, as reported in Flaim, Artif Cells Blood Substit Immobil Biotechnol. 1994; 22(4):1043-54 (doi: 10.3109/10731199409138801), and the absence or insufficient number of pores of Kohn in some species but not others that limit alveoli-to-alveoli ventilation, as reported in Terry et al., Ann Am Thorac Soc. 2016 December; 13(12):2251-2257 (doi: 10.1513/AnnalsATS.201606-448FR), each of which is herein incorporated by reference in its entirety.

To evaluate the effect on injured lungs, the experiment was repeated on a bleomycin-injured rat as generally described Example 5. In brief, 7 days prior to the experiment 0.7 U of bleomycin were nebulized into the trachea using the FLEXIVENT® nebulizer. The nebulizer aerosolizes the liquid and pushes the formed droplets during inspiration resulting in a more diffuse lung distribution. Inspiratory capacity and static compliance were measured as above before and after infusing 100 μL PFB at time 0, while the rat was breathing air. As can be seen in FIG. 7C, unlike in normal rats, there was no decrease in inspiratory capacity or compliance. In fact, both parameters improved slightly over the course of 40 min. These results suggest that the approximately 2 mL of volume added (1 mL PFB gas+1 mL air trapped), filled atelectatic or partially inflated alveoli without affecting the inspiratory capacity and that this filling improved compliance. The experiment may be repeated with graded degrees of lung injuries and different injury models.

### Example 9. Optimize Lung Inflation by Use of Different PFCs and Doses to Maintain Lung Inflation at an Optimal Volume in Rats with Normal or Injured Lungs

The PFC loading dose that is required to increase IC is determined, in which increases in IC define the degree of alveolar recruitment, subsequently increasing compliance and decreasing the work of breathing. PFC gas volume generated is titratable to any desired volume by adjusting the initial infusion dose. The surfactant dysfunction model is used to assess the impact of air trapping on PFC dosing, where lung injury is introduced into rats weighing ˜250 g via nebulizing Tween into the trachea after tracheostomy using the FLEXIVENT® nebulizer to deliver a 5% Tween 20 in saline solution at 0.1, 0.7, or 1.5 mL/kg.

Rats in the Tween groups are connected to the FLEXIVENT® nebulizer and the prescribed dose of Tween is nebulized into the lungs with the rats positioned supine for 30 mins and then prone for 30 mins. PFCs include PFP, C₂BrF₅, PFB and C₃BrF₇ or combinations of two PFCs. PFCs are infused via IV at 10, 40, or 100 μL of emulsion containing 1, 4, or 10 μL of PFC. Control rats receive 100 μL of saline. All rats are connected to the FLEXIVENT® and all lung parameters collected at baseline until rats stabilize, and the prescribed PFC dose is infused at 100 μL/min. Data collection continues for a minimum of 30 minutes or whenever the measurements stabilize, whichever is longer. The FLEXIVENT® allows a terminal maneuver to measure TLC, VC, and RV. Rats are ventilated with 100% O₂ to replace all lung gases and the ventilator stops ventilating to allow consumption of all of the O₂, after which the rat is dead. It cycles the lungs with the chest closed through respiratory maneuvers to calculate TLC, VC, and RV. As a control, 10 normal rats between 175 and 300 g are used to determine weight-adjusted control volumes.

Euthanized rats undergo opening of the chest, tying of the trachea, and removal of the lungs while avoiding lung tears. The lungs are weighed and the volume measured using water displacement. All lungs are frozen at −80° C. in a sealed container. Small lung sections are taken, fixed, sliced, and stained with H&E for histologic analysis which is reviewed by a veterinary pathologist blinded to rat grouping. Degree of injury is graded from 0 (normal) to 5 (severe). Total lung PFC and water content is measured by processing the entire lung.

Weight adjusted FRC is equal to the weight adjusted TLC less the IC measured prior to sacrifice. The volume of air trapped is calculated as the difference between the weight adjusted FRC and total PFB gas volume in the lung. The IC, static, and dynamic lung compliance, and work of breathing collected from the FLEXIVENT® data over time, and the weight adjusted FRC, PFC gas volume, air trapped volume, and lung water content collected postmortem are grouped as a function of Tween dose and PFC dose. Postmortem data is compared to the saline control to assess the effect of PFC dose and to normal rats to assess the effect of Tween dose.

### Example 10. Defining the Impact of Fractional Inspired O₂ on Air Trapping

The impact of inhalation of fractional inspired O₂ (FiO₂) on air trapping is assessed in rats. Two doses of Tween that cause mild and severe lung injury are used as determined from Example 9. PFCs and respective doses that cause minimal and maximal air trapping at the selected Tween doses are used, with saline as a control.

Rats are treated identically as to example 9, except rats are given either 60% FiO₂ or a mixture of 40% gas of the same PFC infused, 50% air, and 10% O₂ to maintain a FiO₂ at 20%. Data are collected as described in example 9.

### Example 11. MicroCT and FLEXIVENT® Assessment of PFC Lung Inflation in Tween, Oleic Acid, and Bleomycin Injury Models

Three lung injury models are used to assess PFC lung inflation following PFC treatment. Using data from Example 9, three concentrations of Tween are selected that induce mild, moderate, or severe changes. Similarly, oleic acid (OA) injury at mild, moderate, and severe levels is induced by administering OA intravenously at 75, 125, and 200 mg/kg 4 hours prior to the study. Additionally, bleomycin injury at mild, moderate, and severe levels is induced by nebulizing 1, 10, or 15 U of bleomycin 7 days prior to the study. Normal rats are treated with the same PFCs, PFC dose, and inhaled gas as the injured rat groups. The PFC and PFC dose that induces reversal of severe injury in the Tween model from Example 9 is utilized.

Gated 3D datasets are acquired at baseline, at peak effect after PFC infusion, and near the end of the observation period. FLEXIVENT® data are collected between microCT scans. The 3D dataset acquired at end expiration and full inspiration at 32 cm H₂O is segmented to isolate the lungs from the chest wall and mediastinum, and lung volumes are calculated by multiplying the number of voxels identified as lung by the voxel volume. The number of darkest pixels +/−2 standard deviation is defined as the volume of aerated lungs. Change in lung volume between expiration and full inflation should be similar to the IC measured with FLEXIVENT®. Subtracting aerated lung volume from total lung volume defines the volume of injured lungs at both expiration and full inflation. The change in volume of injured lungs between the two scans defines the injured alveolar volume that could be inflated. These dependent values are analyzed as described in Example 15.

### Example 12. Defining the PFC Gas Washout Rate to Determine the Maintenance PFC Infusion Rate to Maintain Lung Inflation

Lung function is expected to deteriorate as the vaporized PFC volume is exhaled and lung volume decreases. The change in lung volume and PFC lost in exhaled air over time is determined. Normal rats or rats treated with Tween, OA, or bleomycin at concentrations determined from Examples 9 and 11 are used. The PFC and PFC dose that normalizes lung function after lung injury by Tween while breathing air is used. The experimental procedure is similar to Examples 9-11, except a T-stopper is connected to the tracheostomy tube and the ventilator and a jugular catheter is inserted for blood sampling. IC volume is measured every minute for 5 minutes to establish baseline. The PFC dose is infused, and gas samples are collected every minute for 20 minutes and then every 3 minutes for 60 minutes. To collect a sample, the T-stopper is rotated to close the inhalation port, 0.5 mL of gas sample is aspirated, and the T-stopper is rotated back to maintain breathing. The IC volume after PFC infusion is collected every 5 minutes for 60 minutes. The IC measurement and gas sampling times are adjusted and the correct sampling time is recorded. 30 μL of blood is collected at baseline, and at 1, 3, 5, 10, 20, and 30 minutes for blood PFC content and is frozen at −80° C.

PFC concentrations are measured in each gas and blood sample using gas chromatography. Concentrations in exhaled air are plotted over time and area under the curve as a function of time is calculated. The time at which 10% of the PFC dose is lost is defined for each injury type as well as the half-life elimination rate in the exhaled gas and blood. The change in IC volume as a function of time is analyzed to calculate the IC volume lost/min. Statistical analysis is carried out per Example 15.

### Example 13. Ability for Loading and Maintenance Doses to Keep Injured Lungs Inflated

Normal rats or rats treated with Tween, OA, or bleomycin are used to assess the use of loading and maintenance doses to keep lungs inflated. PFC and its respective loading dose are used as described in Example 12. The maintenance dose is 10% of the loading dose, which is given when 10% of the loading dose is lost in each injury model as determined in Example 12. Rats infused with saline are used as a control.

Rats are prepared and connected directly to the ventilator as described in Examples 9 to 12, and the inhaled gas that causes no air trapping is prepared. Once FLEXIVENT® measurements stabilize, the PFC loading dose is infused and the FLEXIVENT® data is acquired every 3 minutes for 3 hours. 10% of the loading dose is infused to replace 10% of the PFC lost at time intervals as determined from Example 12. IC, compliance, and the work of breathing will from the FLEXIVENT® data are analyzed per Example 15.

### Example 14. Determining the Effect of Pre-Inflated Lungs on Compliance, Work of Breathing, and Blood Gases

The impact of pre-filling lungs is assessed on both lung parameters and blood gases. Normal rats or rats treated with Tween, OA, and bleomycin to produce moderate to severe injury are used. The PFC and respective loading dose that normalize lung parameters in severe lung injury are used as determined by Example 12.

Rats are anesthetized and prepared for the FLEXIVENT® experiment. Rats are given a jugular catheter advanced to the right atrium to assess mixed venous blood gases and pH. A second catheter is inserted in the carotid artery and advanced to the aorta to measure arterial blood gas. Animals are connected to the FLEXIVENT® and the appropriate inhaled gas that does not cause air trapping is connected, with ventilation initiated at 0 cm H₂O PEEP. Baseline IC, compliance, and work of breathing is collected until stabilized. Mixed venous and arterial blood gases are collected and analyzed using a point of care blood gas analyzer. The loading PFC dose is infused and 10% of the loading dose is infused as a maintenance dose at the appropriate time point for each model as defined in Examples 12 and 13 for each lung injury model. Rats infused with an equal loading and maintenance dose of saline serve as controls.

Starting with 9 rats in the Tween injury model, FLEXIVENT® data is stabilized at a new equilibrium, and venous and arterial blood gases are measured. If blood gases are below normal, PEEP is increased by 3 cm H₂O and the experiment repeats until blood gases normalize. This process continues with additional sets of 3 rats with 10% increases in PFC loading doses. If blood gases normalize at 0 PEEP, a new set of 3 rats is studied where the loading dose is decreased by 25% with the maintenance dose adjusted accordingly. The experiment is repeated until blood gases no longer normalize. This cycle is repeated for the OA and bleomycin models.

### Example 15. Statistics

Each parameter of interest is the dependent variable and how it changes from baseline over time is assessed statistically using 2-way ANOVA with time and group as independent variables. Parameters that have statistically significant change following Tuckey correction are evaluated with post-hoc analysis using 2-tailed paired t-test within groups and unpaired t-test between groups. With 6 rats/group 25% change in the mean can be detected and with 12 rats/group 17% change in the mean can be detected if variance equals 15%, at 80% power with an α-error of 5%.

All citations to references, including, for example, citations to patents, published patent applications, and articles, are herein incorporated by reference in their entirety.

The section headings used herein are for organizational purposes only and are not to be construed as limiting the subject matter described in any way.

While various aspects of the invention are described herein, it is not intended that the invention be limited by any particular aspect. On the contrary, the invention encompasses various alternatives, modifications, and equivalents, as will be appreciated by those of skill in the art. Furthermore, where feasible, any of the aspects disclosed herein may be combined with each other (e.g., the feature according to one aspect may be added to the features of another aspect or replace an equivalent feature of another aspect) or with features that are well known in the art, unless indicated otherwise by context.

What is claimed is:

1. A method of treating or preventing a respiratory disease or condition in a subject in need thereof, the method comprising administering an effective amount of a composition comprising one or more perfluorocarbons to the subject.

2. A method of inflating the lungs of a subject in need thereof, the method comprising administering an effective amount of a composition comprising one or more perfluorocarbons to the subject.

3. A method of reducing the positive-end expiratory pressure (PEEP) needed for mechanical ventilation, the method comprising administering an effective amount of a composition comprising one or more perfluorocarbons to the subject, wherein the subject is on mechanical ventilation or is need of mechanical ventilation.

4. The method of any one of the preceding claims, wherein the composition is administered via inhalation of perfluorocarbon gas.

5. The method of claim 4, wherein the perfluorocarbon gas is delivered via an inhaler.

6. The method of claim 4, wherein the perfluorocarbon gas is delivered via a mechanical ventilator.

7. The method of any one of claims 1-3, wherein the composition is administered intravenously.

8. The method of any one of claims 1-3 or 7, wherein the composition comprises nanodroplets each having a perfluorocarbon core comprising the one or more perfluorocarbons.

9. The method of claim 8, wherein the nanodroplets were formed by high pressure microfluidization or sonication at a temperature at which the perfluorocarbon is in liquid form.

10. The method of claim 9, wherein the microfluidization was performed at a pressure between about 2,000 psi and about 23,000 psi.

11. The method of any one of claims 1-3 or 7-10, wherein the composition comprises microbubbles each having a perfluorocarbon core comprising the one or more perfluorocarbons.

12. The method of any one of the preceding claims, wherein the one or more perfluorocarbons comprises perfluorobutane (PFB).

13. The method of any one of the preceding claims, wherein the one or more perfluorocarbons comprises dodecafluoropentane (DDFP).

14. The method of any one of the preceding claims, wherein the one or more perfluorocarbons comprises a mixture of perfluorobutane (PFB) and dodecafluoropentane (DDFP).

15. The method of any one of the preceding claims, wherein the one or more perfluorocarbons comprise perfluoropropane.

16. The method of any one of the preceding claims, wherein the one or more perfluorocarbons comprises 1-bromoheptafluoropropane (C₃BrF₇).

17. The method of any one of the preceding claims, wherein the one or more perfluorocarbons comprises bromopentafluoroethane (C₂BrF₅).

18. The method of any one of claims 1-17, wherein the effective amount is an amount effective to fill the subject's lungs with perfluorocarbon gas to a volume that is sufficient to improve compliance and/or decrease an opening pressure of the subject's lungs, optionally wherein the volume is at least a residual volume (RV) but not more than a functional residual capacity (FRC).

19. The method of claim 18, wherein the effective amount is an amount effective to fill the subject's lungs to less than or up to the functional residual capacity (FRC) with the perfluorocarbon gas.

20. The method of claim 18, wherein the effective amount is an amount effective to fill the subject's lungs to the residual volume (RV) with the perfluorocarbon gas.

21. The method of any one of the preceding claims, wherein the effective amount is an amount effective to fill the lungs to a predetermined volume.

22. The method of claim 21, wherein the predetermined volume comprises a volume of air that is expected to be trapped in the subject's lungs from the administered one or more perfluorocarbons.

23. The method of claim 21, wherein the perfluorocarbon composition comprises nanodroplets, wherein the one or more perfluorocarbons comprise perfluorobutane (PFB) and the effective amount is about 1/150 of the predetermined volume, or wherein the one or more perfluorocarbons comprise perfluoropropane (PFP) and the effective amount is about 1/155 of the predetermined volume.

24. The method of any one of the preceding claims, wherein administering the composition comprises administering an initial loading dose of the composition followed by continuous or intermittent administration of the composition.

25. The method of claim 24, wherein the continuous or intermittent administration of the composition is administered at a rate sufficient to substantially replace perfluorocarbon gas losses from the subject's lungs that occur after the initial loading dose of the composition.

26. The method of claim 24 or 25, wherein the continuous and/or intermittent administration of the composition is administered intravenously.

27. The method of claim 24 or 25, wherein the continuous and/or intermittent administration of the composition is administered as a gas by inhalation.

28. The method of any one of the preceding claims, wherein the subject has a respiratory disease or condition, wherein the respiratory disease or condition is acute lung injury (ALI) or acute respiratory distress syndrome (ARDS).

29. The method of any one of the preceding claims, wherein the subject has coronavirus infection, optionally wherein the coronavirus infection is COVID-19.

30. The method of any one of the preceding claims, wherein the subject has or is experiencing one or more of: ALI, ARDS, neonatal respiratory distress syndrome, sudden acute respiratory syndrome (SARS), infectious lung disease, chemical pneumonitis, aspiration pneumonia, traumatic lung injury, pulmonary fibrosis, interstitial pneumonitis, atelectasis, sedation, and long-term bed rest.

31. The method of any one of the preceding claims, wherein the subject has ALI or ARDS caused by one or more of inflammatory storm, shock, trauma, sepsis, pneumonia, aspiration, burns, major surgery, blood transfusion, pancreatitis, or severe viral infection, optionally a coronavirus infection (e.g., COVID-19).

32. The method of any one of the preceding claims, wherein the method reduces an opening pressure of the lungs of the subject.

33. The method of any one of the preceding claims, wherein the subject has surfactant deficiency and/or dysfunction, and wherein the method improves surfactant deficiency and/or dysfunction in the subject.

34. The method of any one of the preceding claims, wherein the method increases compliance of alveoli of the subject.

35. The method of any one of the preceding claims, wherein the method causes inflation of the lungs of the subject.

36. The method of claim 35, wherein the inflation is at least partially caused by air trapping.

37. The method of any one of the preceding claims, wherein the method reduces or prevents collapse of alveoli at the end of exhalation in the subject.

38. The method of any one of the preceding claims, wherein the method reduces or prevents atelectasis in the subject.

39. The method of any one of the preceding claims, wherein the method reduces or prevents inflammation in the lungs of the subject.

40. The method of any one of the preceding claims, wherein the method reduces or prevents pulmonary edema in the subject.

41. The method of any one of the preceding claims, wherein the method enhances oxygen delivery and/or gas exchange in the alveoli of the subject.

42. The method of any one of the preceding claims, wherein the method decreases ventilation-perfusion mismatch (V-Q mismatch) in the lungs of the subject, optionally wherein the method normalizes DLCO in the lungs of the subject.

43. The method of any one of the preceding claims, wherein the method improves diffusing capacity (DLCO) in the lungs of the subject.

44. The method of any one of the preceding claims, wherein the method improves or normalizes of one or more of blood O₂ concentration, blood CO₂ concentration, blood pH, and tissue O₂ concentration in the subject.

45. The method of any one of the preceding claims, wherein the method reduces the length of time the subject is on supplemental oxygen or prevents the subject from requiring supplemental oxygen.

46. The method of any one of the preceding claims, wherein the method reduces or prevents one or more symptoms associated with the respiratory disease or condition in the subject.

47. The method of claim 46, wherein the one or more symptoms are selected from the group consisting of dyspnea, wheezes, chest tightness, and cough.

48. The method of any one of the preceding claims, wherein the method improves exercise tolerance in the subject.

49. The method of any one of the preceding claims, wherein the method reduces the length of time or frequency the subject is hospitalized or admitted to an ICU or prevents the subject from being hospitalized or admitted to an ICU.

50. The method of any one of the preceding claims, wherein the subject is undergoing or in need of mechanical ventilation.

51. The method of any one of the preceding claims, wherein the method reduces FiO₂ needed for adequate oxygenation in the subject.

52. The method of any one of the preceding claims, wherein the subject is on mechanical ventilation at the time of the administration of the composition.

53. The method of any one claims 1-51, wherein the subject is not on mechanical ventilation at the time of the administration of the composition, the method further comprising initiating mechanical ventilation after the administration of the composition, optionally wherein the PEEP is zero.

54. The method of any one of claims 50-53, wherein the method reduces an amount of positive-end expiratory pressure (PEEP) needed for adequate oxygenation and ventilation in the subject.

55. The method of claim 54, wherein the method reduces PEEP to zero.

56. The method of any one of claims 50-55, further comprising weaning the subject from mechanical ventilation.

57. The method of any one of claims 50-56, wherein the method mitigates the extent of ventilator-induced lung injury (VILI) or ventilator-associated lung injury (VALI) in a subject.

58. The method of any one of claims 50-57, wherein the method prevents the subject from developing VILI or VALI.

59. The method of any one of claims 50-58, wherein the method reduces the length of time the subject is placed on mechanical ventilation.

60. The method of any one of claims 1-50, wherein the subject is not on mechanical ventilation at the time of the administration of the composition, and wherein the method prevents the subject from requiring mechanical ventilation.

61. The method of any one of the preceding claims, further comprising administering to the subject an inhaled gas comprising a perfluorocarbon, wherein the administration of the inhaled gas results in at least partially reversing inflation caused by an intravenously administered composition comprising one or more perfluorocarbons.
